(19) United States
(12) Patent Application Publication
(10) Pub. No.: US 2023/0233230 A1
(43) Pub. Date: Jul. 27, 2023

(54) DEVICE AND METHOD FOR OOCYTE RETRIEVAL FROM FALLOPIAN TUBES

(71) Applicant: MAGNA MATER MEDICAL LTD., Tel-Aviv (IL)

(72) Inventors: Gil BACHAR, Tel-Aviv (IL); Ekaterina SHLUSH, Herzelia (IL); Yotam AMIRAM, Tel-Aviv (IL); Josef SHACHRUR, Parades Hana Karkur (IL); Ben AZOURI, Tel-Aviv (IL); Ishai ISRAEL, Haifa (IL)

(73) Assignee: MAGNA MATER MEDICAL LTD., Tel-Aviv (IL)

(21) Appl. No.: 18/009,074

(22) PCT Filed: Jun. 9, 2021
(86) PCT No.: PCT/IL2021/050693

§ 371 Date: Dec. 8, 2022

Related U.S. Application Data
(60) Provisional application No. 63/111,767, filed on Nov. 10, 2020.
(60) Provisional application No. 63/037,019, filed on Jun. 10, 2020.

(51) Int. Cl. A61B 17/435; A61M 25/10; A61M 1/00; A61M 25/09; A61M 25/01; A61B 17/12

(52) U.S. Cl. CPC A61B 17/435; A61M 25/10; A61M 1/84; A61M 25/09; A61M 25/0105; A61B 17/1204; A61B 17/12099; A61B 17/12136; A61B 2217/005; A61B 2217/007

(57) ABSTRACT

An oocytes or ovum retrieval device is disclosed. The device may include a retrieval catheter dimensioned for transvaginal insertion into a cervical canal and for advancing distally into a fallopian tube and the ampulla of a female mammal. The retrieval catheter may include one or more fluid delivery lumens having a fluid delivery port for delivering a flushing fluid into said fallopian tube, an at least one aspiration opening in fluid connection with an internal aspiration lumen of said retrieval catheter, wherein the size of the at least one aspiration opening is determined based on a size of oocytes or ovum.

### CROSS REFERENCE TO RELATED APPLICATIONS

This application is a PCT patent application which claims the benefit of priority under 35 U.S.C. § 119(e) of U.S. Provisional Application No. 63/111,767, filed on Nov. 10, 2020 and U.S. Provisional Application No. 63/037,019, filed on Jun. 10, 2020. The contents of the above applications are incorporated by reference as set forth herein in their entirety.

### FIELD OF THE INVENTION

Some embodiments of the present invention relate in general to retrieval of oocytes or ovum from a subject. More specifically, the present invention relates to a single device for retrieval of oocytes or ovum from a subject.

### BACKGROUND

In Vitro Fertilization (IVF) is a common intervention to assist people suffering from childlessness. First, the female subject is provided with any required hormonal stimulation, to increase number of follicles being matured in the female. Then, the follicles are emptied while still in the ovaries. This is normally done transvaginally, which means that an oocyte retrieval needle is used to penetrate the vaginal wall and the ovaries. Following localization of the follicles by ultrasound, an oocyte retrieval needle is used to puncture and enter each follicle, and then the follicular fluid containing the oocytes is retrieved by aspiration. The retrieval is achieved through an induced negative pressure. In cases where the oocytes are not released from the follicle through aspiration only, a pre-warmed flush solution may be used together with the aspiration to release the oocytes and to increase the aspirated fluid volume.

This procedure, which is typically performed under sedation, is lengthy and time consuming, and may be associated with injury to pelvic organs, hemorrhage, infection, as well as pain and discomfort to the patient. Additional complications may result from the administration of intravenous sedation or general anesthesia.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

Some aspects of the invention may be directed to an oocytes or ovum retrieval device comprising: a retrieval catheter dimensioned for transvaginal insertion into a cervical canal and\or for advancing distally into a fallopian tube and\or the ampulla of a female mammal. In some embodiments, the retrieval catheter includes:
(i) one or more fluid delivery lumens having a fluid delivery port for delivering a flushing fluid into the fallopian tube, and
(ii) at least one aspiration opening in fluid connection with an internal aspiration lumen of the retrieval catheter, wherein the size of the at least one aspiration opening is determined based on a size of oocytes or ovum.

In some embodiments, the device may further include an introductory catheter dimensioned for transvaginal insertion into a cervical canal of a female mammal, such that the retrieval catheter is dimensioned for slidably advancing within a lumen of the introductory catheter along a longitudinal axis thereof, such that a distal end of the retrieval catheter extends distally of a distal tip of the introductory catheter and into the fallopian tube.

In some embodiments, the distal end of the retrieval catheter extends distally of the distal tip of the introductory catheter and the at least one aspiration opening is distal to the distal tip.

In some embodiments, the device may further include a first occluding element for deployment at an occlusion point, such that, the first occlusion element is configured to prevent passage of the fluid distally of the occlusion point in the fallopian tube. In some embodiments, the device may further include a second occluding element for deployment at an occlusion point proximally of the at least one fluid delivery port, such that, the second occlusion element is configured to prevent passage of the fluid proximally of the occlusion point in the fallopian tube. In some embodiments, at least one of the first occluding element and the second occluding element is an inflatable balloon.

In some embodiments, at least a distal portion of a length of the retrieval catheter is bendable.

In some embodiments, the device may further include one or more fluid reservoirs in fluid connection with the one or more fluid delivery lumens. In some embodiments, the device may further include a vacuum source in fluid connection with the internal aspiration lumen.

In some embodiments, an outer diameter of at least a distal portion of a sidewall of the retrieval catheter is between 1 and 1.4 mm. In some embodiments, an outer diameter of at least a distal portion of a sidewall of the retrieval catheter is between 1.2 and 1.8 mm. In some embodiments, the retrieval catheter has a proximal portion that defines a diameter which is larger than the distal portion. In some embodiments, at least one cross-sectional dimension of the internal aspiration lumen of the retrieval catheter is larger than 0.4 mm. In some embodiments the wall thickness of aspiration lumen is in the range of 50 um to 200 um.

In some embodiments, the internal aspiration lumen of the retrieval catheter is dimensioned such that a maximal inscribed circle within a cross-section thereof has a diameter that is larger than 0.4 mm. In some embodiments, the retrieval catheter includes another internal lumen, and a ratio between a cross sectional dimension of the internal aspiration lumen and a cross sectional dimension of the other internal lumen is at least 2 to 1.

In some embodiments, the retrieval catheter includes a plurality of the aspiration openings arranged about an external sidewall of the retrieval catheter. In some embodiments, the internal aspiration lumen and the fluid delivery lumen are arranged in a helically wound arrangement along a length of the retrieval catheter. In some embodiments, the helically wound arrangement defines a hollow passage extending internally along a length of the retrieval catheter, and wherein the hollow passage is dimensioned to receive a guidewire therethrough.

In some embodiments, the retrieval catheter includes an inflation lumen, and wherein the inflation lumen is in fluid connection with at least one of the first occluding element and the second occluding element. In some embodiments, the first occluding element includes at least one fluid delivery port for delivering a flushing fluid into the fallopian tube. In some embodiments, the retrieval catheter includes a pressure beaching internal lumen, and the pressure beaching internal lumen is configured to breach an opening is the sidewall of the retrieval catheter when a threshold pressure is reached within the second internal lumen.

In some embodiments, the retrieval catheter includes another fluid delivery lumen, and the other fluid delivery lumen includes an internal unidirectional valve to prevent passage of fluid internally of the other fluid delivery lumen prevent passage of the fluid of the unidirectional valve.

Some additional aspects of the invention may be directed to a method for retrieval of oocytes or ovum from a subject comprising:
providing a device comprising:
   a retrieval catheter dimensioned for transvaginal insertion into a cervical canal and for advancing distally into a fallopian tube and the ampulla of a female mammal,
   wherein the retrieval catheter includes:
   (i) one or more fluid delivery lumens, each having a fluid delivery port for delivering a flushing fluid into the fallopian tube, and
   (ii) at least one aspiration opening in fluid connection with an internal aspiration lumen of the retrieval catheter, wherein the size of the at least one aspiration opening is determined based on a size of oocytes or ovum;
advancing the retrieval catheter distally into the fallopian tube and the ampulla;
delivering a flushing fluid through the distal fluid delivery port into the fallopian tube; and
aspirating the flushing fluid and entrained oocytes or ovum therein through the at least one aspiration opening.

In some embodiments, the method may further include, occluding a passage of the fluid, distally from the fluid delivery port, by inserting a first occluding element, included in the device, for deployment of a first occlusion point in the fallopian tube. In some embodiments, the method may further include, occluding the passage of the fluid, proximity to the fluid delivery port, by inserting a second occluding element, included in the device, for deployment of a second occlusion point in the fallopian tube.

Some aspects of the invention may be directed to another oocytes or ovum retrieval device comprising:
a retrieval catheter dimensioned for transvaginal insertion into a cervical canal and for advancing distally into a fallopian tube and the ampulla of a female mammal, wherein the retrieval catheter includes one or more fluid delivery lumens having a fluid delivery port for delivering a flushing fluid into the fallopian tube, and
an introductory catheter dimensioned for entering a uterus of a woman or a female mammal transcervicaly, wherein the introductory catheter comprised an aspiration catheter lumen and at least one aspiration opening in fluid connection with the aspiration lumen of the introductory catheter, wherein the size of the at least one aspiration opening is determined based on a size of oocytes or ovum.

In some embodiments, the retrieval catheter may be inserted in the introductory catheter lumen. In some embodiments, the introductory catheter is dimensioned to occlude liquid exiting the fallopian tube to the uterus.

In some embodiments, the device may further include a first occluding element for deployment at an occlusion point, such that, the first occlusion element is configured to prevent passage of the fluid distally of the occlusion point in the fallopian tube. In some embodiments, the device may further include a second occluding element for deployment at an occlusion point proximally of the at least one fluid delivery port, such that, the second occlusion element is configured to prevent passage of the fluid proximally of the occlusion point in the fallopian tube. In some embodiments, at least one of the first occluding element and the second occluding element is an inflatable balloon.

In some embodiments, at least one of the one or more fluid delivery lumens is designed to deliver of gas or liquid for inflating the balloon. In some embodiments, the same fluid delivery lumen is configured to deliver the flushing fluid and gas or liquid for inflating the balloon. In some embodiments, at least a distal portion of a length of the retrieval catheter is bendable.

In some embodiments, the device may further include a fluid reservoir in fluid connection with the at least one fluid delivery lumen. In some embodiments, the device may further include a vacuum source in fluid connection with the internal aspiration lumen.

Some additional aspects of the invention may be directed to a method for retrieval of oocytes or ovum from a subject comprising:
providing a device comprising:
   a retrieval catheter dimensioned for transvaginal insertion into a cervical canal and for advancing distally into a fallopian tube and the ampulla of a female mammal, wherein the retrieval catheter includes one or more fluid delivery lumens having a fluid delivery port for delivering a flushing fluid into the fallopian tube, and
   an introductory catheter dimensioned for entering a uterus of a woman or a female mammal transcervicaly, wherein the introductory catheter comprised an aspiration catheter lumen and at least one aspiration opening in fluid connection with the aspiration lumen of the introductory catheter, wherein the size of the at least one aspiration opening is determined based on a size of oocytes or ovum;
advancing the introductory catheter into a uterus;
advancing the retrieval catheter distally into the fallopian tube and the ampulla;
   delivering a flushing fluid through the distal fluid delivery into the fallopian tube; and
aspirating the flushing fluid and entrained oocytes or ovum therein through the at least one aspiration opening.

In some embodiments, the method may further include, occluding a passage of the fluid, distally from the fluid delivery port, by inserting a first occluding element, included in the device, for deployment of a first occlusion point in the fallopian tube. In some embodiments, the method may further include, occluding the passage of the fluid, proximity to the fluid delivery port, by inserting a second occluding element, included in the device, for deployment of a second occlusion point in the fallopian tube.

Some additional aspects of the invention may be directed to method for retrieving oocytes from a fallopian tube of a woman comprising:
a. Advancing a retrieval catheter transcervicaly into the fallopian tube.
b. Delivering of a flushing fluid into the fallopian tube from a delivery port in said catheter.
c. Aspirating said flushing fluid.

Some additional aspects of the invention may be directed to a device comprising a catheter dimensioned for entering a fallopian tube of a woman in a transcervical procedure, the said catheter comprising:
a fluid delivery lumen;
a fluid delivery port in a fluid connection with the fluid delivery lumen, the delivery port allowing the delivery of a flushing liquid into the fallopian tube;
a balloon, in a fluid connection with the fluid delivery lumen designed to occlude the fallopian tube from fluids at an occlusion point;
wherein the device allows the inflation of the balloon without spilling of liquid into the fallopian tube and the device further allows the spilling of flushing fluid into the fallopian tube without deflating the balloon.

In some embodiments, the delivery port may be located on the catheter. In some embodiments, the delivery port may be located on the balloon.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1 shows a female reproductive system, which includes (in order, from the to the exterior to the interior) the vagina 151, the cervix 152, the uterus 153, two fallopian tubes 154 and two ovaries 155.

FIG. 2A shows an initial position 251, in which retrieval catheter 204 does not extend externally from introductory catheter tip 210 in the distal front part (toward the patient's body), while on the proximal side, retrieval catheter 204 extends externally from introductory catheter back part 207, a section marked as retrieval catheter back part 211 according to some embodiments of the invention.

FIG. 2B shows retrieval catheter in a retrieval position 252, also referred herein as the retrieval position 252 according to some embodiments of the invention.

FIG. 2C shows device 200 inside uterus 153 according to some embodiments of the invention.

FIG. 2D shows a tilt operation marked with arrow 261 according to some embodiments of the invention.

FIG. 2E shows another embodiment of device 200 where introductory catheter 202 has a rounded distal part.

FIG. 2F shows yet another embodiment of device 200 where the introductory catheter 202 includes an introductory catheter center rigid part 212, an introductory catheter front rigid part 216, and a bendable part 218.

FIG. 2G shows yet another embodiment of device 200 where retrieval catheter 204 includes fluid delivery lumen 806 having a fluid delivery port 808 and a retrieval catheter aspiration opening 209 in fluid communication with an internal aspiration lumen 208.

FIG. 2H shows the embodiment of device 200 illustrated in FIG. 2G during a retrieval procedure.

FIGS. 3A and 3B shows flowcharts of methods for oocytes or ovum retrieval, according to some embodiments of the present invention.

FIG. 4A shows one example of modified introductory catheter 4202a which is similar to introductory catheter 202 with the addition of camera 402 manifested as miniature digital camera located on introductory catheter tip 210 according to some embodiments of the invention.

FIG. 4B shows another example of modified introductory catheter 4202b which is similar to introductory catheter 202 with the addition of camera 402 manifested as a digital camera located outside of the female reproductive system and near the rear part of introductory catheter 4202b according to some embodiments of the invention.

FIG. 5 shows modified retrieval catheter 5204 according to some embodiments of the invention.

FIG. 6 shows a cross section of optical fiber 502 according to some embodiments of the invention.

FIGS. 7A-C show exemplary oocytes or ovum retrieval device 700. Device 700 may be included in device 200 described above according to some embodiments of the invention.

FIGS. 8A-8B, 9A-9B, 10A-10B, 11A-11C, 12A-12I, 13A-13D, 14A-14G, 15A-15D, 16A-16B, 17A-17C, 18, 19A-19B, 20A-20B, 21A-21B, 22A-22C, 23A-23D and 24A-24C show other examples of an oocyte retrieval device according to some embodiments of the invention.

### DETAILED DESCRIPTION

Disclosed herein are devices and methods for oocyte and/or ovum retrieval from the fallopian tubes, and, in particular, from the fallopian tube ampulla, in human and/or other mammal subjects. The devices presented may be used in a transvaginal and\or transcervical procedure. In some embodiments, the present device provides for retrieval of oocytes or ovum after ovulation and natural migration to the fallopian tube. Retrieved oocytes or ovum may then be used for any known purpose, and, in particular, in IVF procedures and\or for fertility preservation. In this entire disclosure, the terminology of “oocytes” or “ovum” may be exchanges freely, wherein wherever one term is used the other term is included without the need for explicit listing. As used herein, the term “transvaginal” refers to a procedure done in the reproductive system of a female delivered via the vagina. As used herein the term “transcervical” refer to a procedure performed by way of the uterine cervix.

In some embodiments, the present disclosure provides for a device comprising an introductory catheter configured and dimensioned for inserting and/or placing into a female reproductive device, e.g., transvaginally into the cervical canal, for delivery of a distal end thereof in proximity to a fallopian tube isthmus.

In some embodiments, an inner retrieval catheter may be introduced into the introductory catheter lumen, and advanced internally thereof into an oocyte retrieval position inside the fallopian tube and the ampulla, wherein a distal portion of the inner retrieval catheter extends distally from a distal end of the introductory catheter. In some embodiments, the inner retrieval catheter may be advanced, e.g., to an ampulla region of the fallopian tube.

In some embodiments, a device of the present disclosure does not include an introductory catheter, wherein the retrieval catheter may be introduced and advanced directly into an oocyte retrieval position inside the fallopian tube and the ampulla.

In some embodiments, the device may include one or more fluid delivery lumens having a fluid delivery port for delivering a flushing fluid into the fallopian tube, e.g., a liquid and/or a gas, e.g., a saline solution, saline with heparin, flushing medium, oocyte retrieval medium, etc., which functions to release and flush oocytes and/or ovum proximally, within a retrieval region defined by the present device.

In some embodiments, the retrieval catheter may further comprise one or more aspiration openings about a longitudinal dimension of a distal portion thereof which extends past a distal end of the introductory catheter and proximally to a distal end of the inner retrieval catheter. In some embodiments, the size of the at least one aspiration opening may be determined based on a size of oocytes or ovum. In some embodiments, the one or more aspiration openings may be in fluid connection with a lumen disposed internally of the inner retrieval catheter. In some embodiments, oocytes and/or ovum being flushed proximally from their position in the fallopian tube may be suctioned into the one or more aspiration openings by a suitable suctioning function of the present device operating, e.g., from a proximal region of the present device, for ultimate collection at a proximal opening of the retrieval catheter, for use in conjunction with any known IVF procedure and\or egg freezing and\or egg donation and\or research and\or other purposes.

In some embodiments, the present device may further comprise one or more occluding elements, e.g., one or more inflatable balloons and\or one or more expandable structures, configured for occluding one or more points within the fallopian tube, e.g., at the isthmus, the ampulla, the infundibulum, the fimbria, and/or another location. In some embodiments, the one or more occluding elements work to define an oocyte and/or ovum retrieval region of the present device between one or more occlusion points defined thereby. In some embodiments, the occlusion points limit and/or prevent oocytes and/or ovum from being flushed past an occluding element distally and/or proximally of the defined retrieval region. In some embodiments, the one or more occluding elements limit and/or prevent flushing oocytes and/or ovum only distally, past a defined occlusion point which is distal to the oocyte and/or ovum retrieval region defined by the present device within the fallopian tube. In some embodiments, the one or more occluding elements define an occlusion region which limits and/or prevents flushing oocytes and/or ovum both distally and proximally of the defined oocyte and/or ovum retrieval region. In some embodiments, the one or more occluding elements may function to retain any flushing fluids within a defined retrieval region and prevent passage of fluids distally and/or proximally, past any occluding element.

In some embodiments, the present device may comprise an imaging system configured for providing an image stream, e.g., video stream, for providing guidance to the medical practitioner operating the present device. In some embodiments, the present device may be operated in conjunction with an external imaging system e.g., an abdominal or vaginal sonar imaging system (also known as ultrasound imaging system abbrev. US), magnetic resonance imaging (MM) system, X-ray imaging system, and the like.

In some embodiments, the present device may be configured for oocytes retrieval from the female reproductive system in any mammal, for example, the reproductive system of a woman. In some embodiments, the present device may be configured for oocytes retrieval from either or both fallopian tubes within the female reproductive system.

In some embodiments, any device disclosed herein may be useful for delivering fluids and/or aspiration of fluids from any lumen in a human or animal body. In such case the size and dimensions of the lumens may be determined according to the required lumen, for example, a blood vessel, urethra, nasal, etc.

FIG. 1 shows a female reproductive system, which includes (in order, from the to the exterior to the interior) the vagina 151, the cervix 152, the uterus 153, two fallopian tubes 154 and two ovaries 155.

In the following description the term ‘proximal’ is used to describe an element positioned closer to the uterus side, relative to other elements, and the term ‘distal’ is used to describe an element positioned closer to the ovary side, relative to other elements. In the following description, sizes of elements and components in proposed devices and methods have been chosen to match human reproductive system. It should be understood that obvious variations in size of such elements and components may occur in the proposed device and methods to accommodate oocyte retrieval in non-human mammals. All figures are not to scale unless mentioned specifically.

Each fallopian tube 154 includes (in order, from proximal to distal) the isthmus 159, the ampulla 158, the infundibulum 157, and the fimbria 156.

In some cases, the procedure detailed below for oocyte retrieval may be preceded by drug administration with stimulation protocols known in the art. Other procedures and protocols for ovulation promotions, as may be known in the art, may be used). The protocol administration promotes ovulation or multiple ovulations from one or both ovaries, followed by oocytes transport into fallopian tubes 154, and, in particular, ampullas 158. In some cases, the methods and devices described below may be preceded by a natural ovulation cycle in which, normally, a single oocyte migrates to the fallopian tube (e.g., in human patients).

In the following, it is assumed that oocytes 160 retrieval may be done in one of the two fallopian tubes 154, or from both of the fallopian tubes 154 sequentially or in parallel. In some cases, the text describes a single side procedure, but a generalization to a two-sided procedure is implicit for all examples.

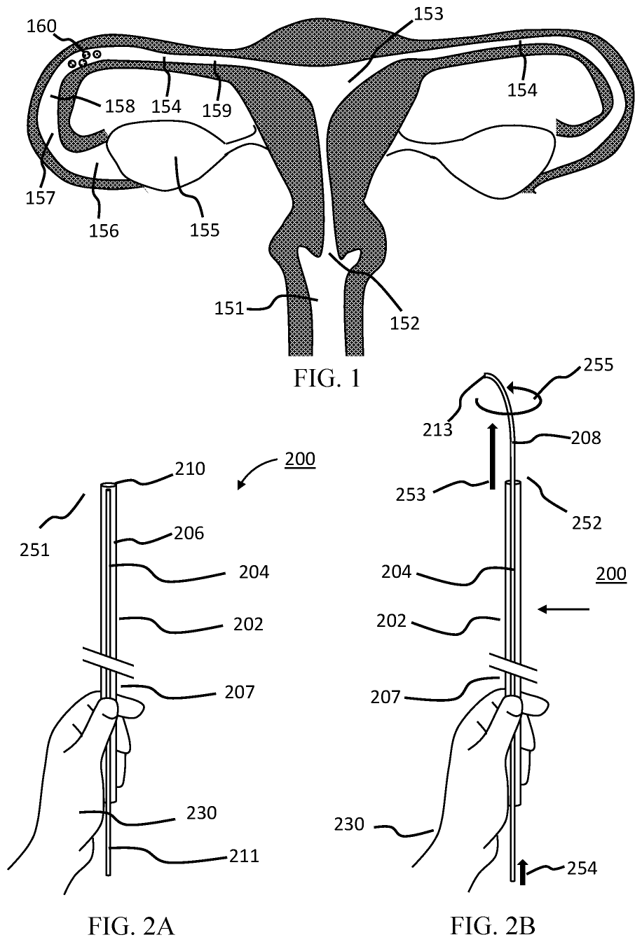

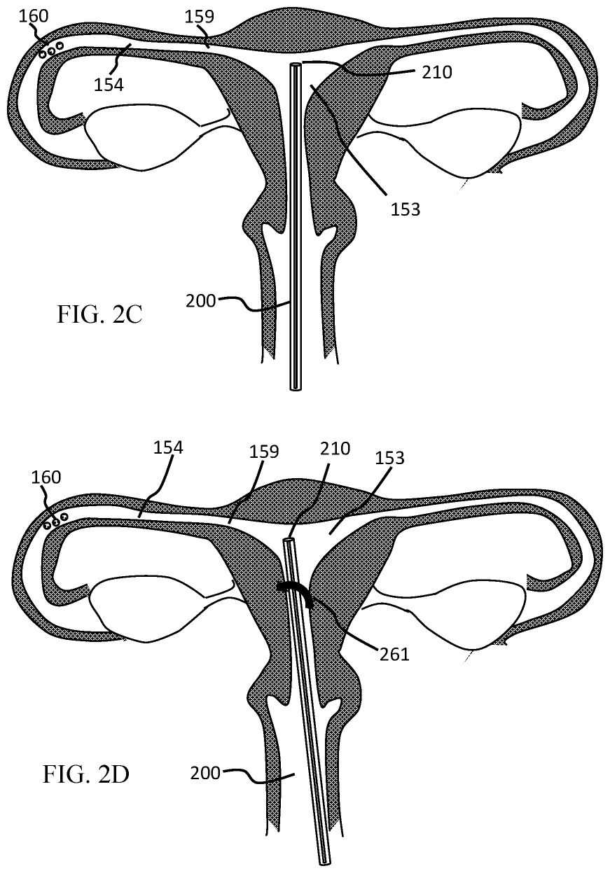

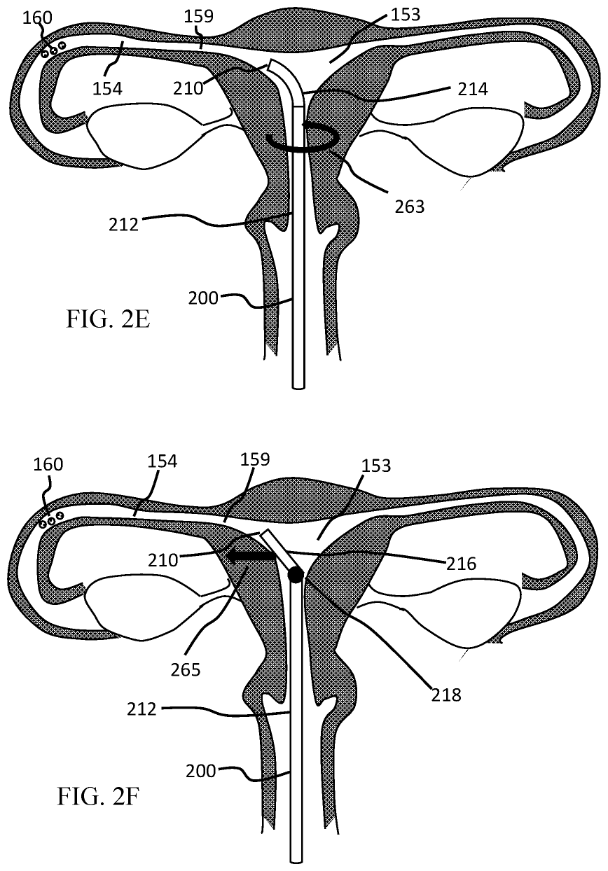

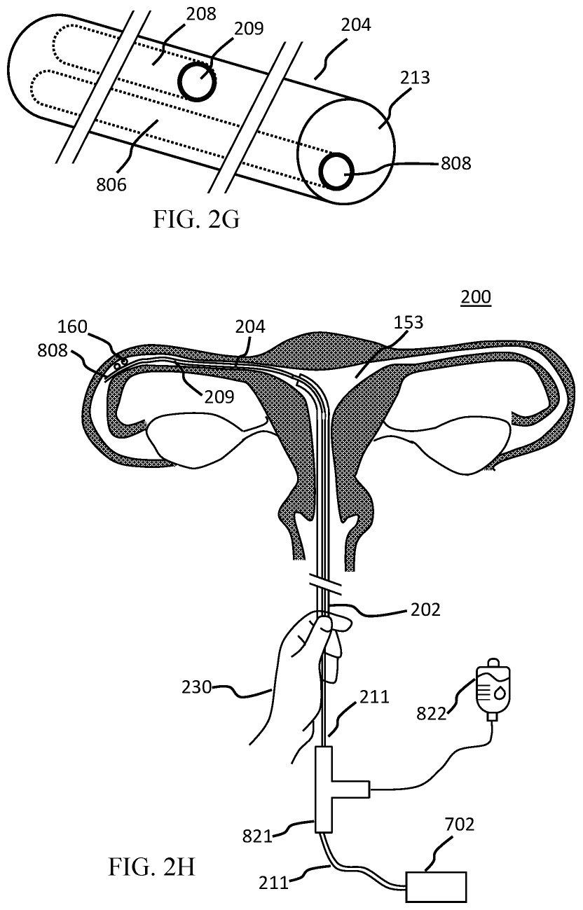

FIGS. 2A-B show device 200. Device 200 is designed to retrieve oocytes 160 from fallopian tubes 154 (FIG. 1). In some embodiments, device 200 is designed to enter female reproductive system in transcervical and\or transvaginal procedure. In an example, device 200 is shown as held by an operator's hand 230 (e.g., by medical staff, medical doctor, etc.), In some embodiments, device 200 may be held by a machine, robot, the patient herself, attached to another device, attached to patient body, and the like. In some of the following figures operator's hand 230 is shown to illustrate all or some of these optional holding or mounting options.

In an embodiment, device 200 may include an introductory catheter 202, which is made of (or covered with) biocompatible material, for example, polytetrafluoroethylene (PTFE), fluorinated ethylene propylene (FEP), polyether ether ketone (PEEK), Teflon, nylon, polyethylene, polystyrene, titanium, gold, etc. Introductory catheter 202 may be rigid or semi-rigid (e.g., slightly flexible) or soft (flexible and bendable). Introductory catheter 202 includes introductory catheter back part 207 which is kept outside of female reproductive system as detailed below. Introductory catheter 202 may include, e.g., a grip, handle, fitting, or a like (which are not shown) to allow holding or gripping of introductory catheter back part 207 as detailed above. In some cases, holding or gripping of introductory catheter 202 may be done directly on introductory catheter back part 207. Introductory catheter 202 further includes an introductory catheter tip 210, which may be inserted into female reproductive system via the cervix canal, and into uterus 153, as detailed in methods described below.

Introductory catheter 202 typically has a diameter of few millimeters (mm), (in one example 2-20 mm, in one example 1-5 mm), such that introductory catheter 202 can enter uterus 153 from cervix 152. In some embodiment a dilator and\or an obturator, and\or irrigation (which are not presented) may be used to assist to the entrance of introductory catheter 202 into uterus 153. In an embodiment, introductory catheter 202 defines a longitudinal lumen or passage internally thereof, or any similar formation. In FIGS. 2A-B, such a lumen is marked as introductory catheter lumen 206. In some cases, introductory catheter lumen 206 may be used for the entrance of fluids or gases into female reproductive system. In some embodiments, introductory catheter lumen 206 may be used to retrieve fluids or gases or cells from female reproductive system. In some cases, introductory catheter lumen 206 may be used for the introduction of working tools (not seen in the figures) such as tweezers, scissors, etc. into the female reproductive system.

Reference is now made to FIG. 2G which is an illustration of device 200 which includes a retrieval catheter 204. In some embodiments, retrieval catheter 204 may be made of (or covered by) a soft (non-rigid) biocompatible material (e.g., silicone, PTFE, polyurethane, Teflon, etc.).

In some embodiments, retrieval catheter 204 may be dimensioned for transvaginal insertion into a cervical canal and for advancing distally into a fallopian tube and the ampulla of a female mammal. For example, an outer diameter of at least a distal portion of a sidewall of retrieval catheter 204 catheter may be between 1 and 1.4 mm or between 1.2 and 1.8 mm. In some embodiments, these dimensions may be suitable for a oocytes or ovum retrieval device for the use in human females.

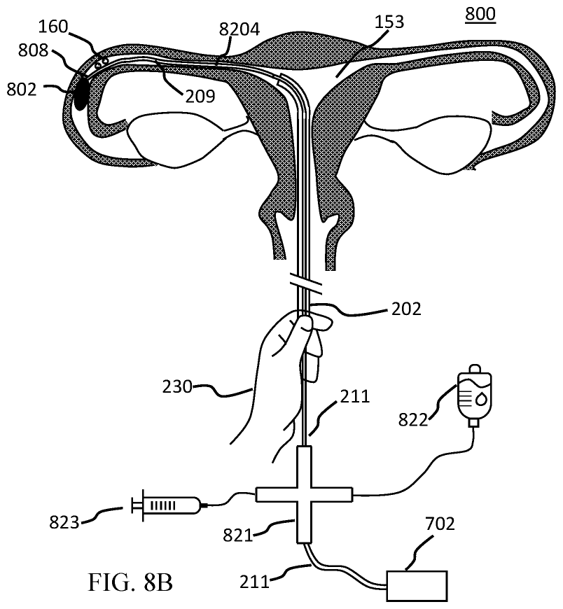

In some embodiments, retrieval catheter 204 may include one or more fluid delivery lumens 806 having a fluid delivery port 808 for delivering a flushing fluid into the fallopian tube (also illustrated in FIG. 8A). In some embodiments, retrieval catheter 204 may include at least one retrieval catheter aspiration opening 209 in fluid connection with an internal aspiration lumen 208 of retrieval catheter, 204, such that the size of at least one retrieval catheter aspiration opening 209 may be determined based on a size of oocytes or ovum (illustrated in FIG. 2H). In some embodiments, the internal aspiration lumen 208 may be the same as fluid delivery lumen(s) 806.

In some embodiments, at least one cross-sectional dimension of the internal aspiration lumen 208 and/or fluid delivery lumen 806 may be larger than 0.4 mm. In some embodiments, the internal aspiration lumen 208 may be dimensioned such that a maximal inscribed circle within a cross-section thereof has a diameter that is larger than 0.4 mm. In some embodiments, when retrieval catheter 204 includes two internal lumens (e.g., fluid delivery lumen and internal aspiration lumen) a ratio between cross sectional dimensions of the fluid delivery lumen and any other internal lumen is at least 2 to 1.

In some embodiments, device 200 may include retrieval catheter 204 dimensioned for transvaginal insertion into a cervical canal and for advancing distally into a fallopian tube 154 and the ampulla 158 of a female mammal (as shown in FIG. 2H), having one or more fluid delivery lumens 806 having a fluid delivery port 808 for delivering a flushing fluid into the fallopian tube. In some embodiments, such a device 200 further includes introductory catheter 202 dimensioned for entering a uterus of a woman or a female mammal transcervicaly, having an aspiration catheter lumen (e.g., introductory catheter lumen 206) and at least one aspiration opening (located in proximity to introductory catheter tip 210) in fluid communication with internal aspiration lumen 208 (e.g., introductory catheter lumen) of introductory catheter 202. In some embodiments, the size of the at least one aspiration opening is determined based on a size of oocytes or ovum.

In an embodiment, retrieval catheter 204 may be inserted in introductory catheter lumen 206, directly or using some conventional fitting, which is not shown. In an embodiment, retrieval catheter 204 may be inserted directly into the female reproductive system 150 without the need of introductory catheter 202. In some embodiments, device 200 may include only retrieval catheter 204.

Referring back to FIG. 2A to show an initial position 251, in which retrieval catheter 204 does not extend externally from introductory catheter tip 210 in the distal front part (toward the patient's body), while on the proximal side, retrieval catheter 204 extends externally from introductory catheter back part 207, a section marked as retrieval catheter back part 211.

In FIG. 2A, initial position 251 is seen such that retrieval catheter 204 is close to introductory catheter tip 210. In some embodiments, at initial position 251, retrieval catheter 204 may be positioned within introductory catheter lumen 206 such that a tip thereof is a few centimeters (cm) (e.g., 1-5 cm) proximally of introductory catheter tip 210, or a few cm (e.g., 1-5 cm) distally of introductory catheter lumen 206. In some embodiments, at initial position 251, retrieval catheter 204 may be positioned outside of introductory catheter lumen 206. In an embodiment, retrieval catheter 204 may be moved axially proximally and distally inside introductory catheter lumen 206, and relative to introductory catheter 202, in a direction marked by arrow 253. In an example, the actuation of retrieval catheter 204 relative to introductory catheter may be done manually (e.g., by the operator), using an actuator (e.g., an electrical motor connected to retrieval catheter back part 211), or in any similar means, by pushing retrieval catheter back part 211 as shown by arrow 254.

In an embodiment, retrieval catheter 204 may be rotated inside introductory catheter lumen 206 and relative to introductory catheter 202, in a direction marked by arrow 255 and vice versa. FIG. 2B shows retrieval catheter in a retrieval position 252, also referred herein as the retrieval position 252. In retrieval position 252, retrieval catheter 204 protruded introductory catheter tip 210 by few cm, in an example 1-15 cm, as explained further below. Retrieval catheter 204 typically has a diameter of 2 mm or less (in an example 0.2-2 mm, in an example 1.1-1.5 mm), allowing it to enter fallopian tube 154. Retrieval catheter 204 includes a retrieval catheter tip 213.

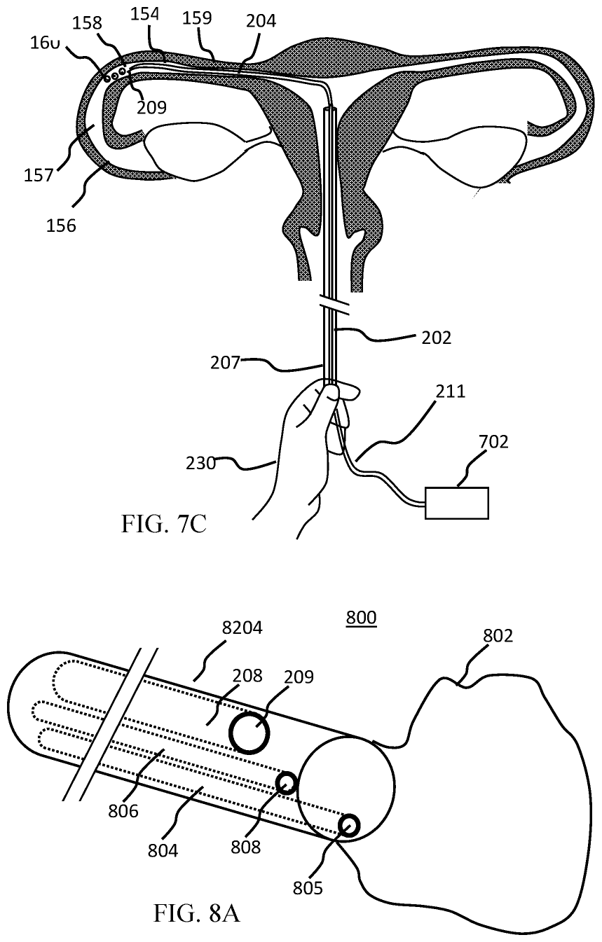

In one example, retrieval catheter 204 has a retrieval catheter cavity, a hole, a slit, a passage, a lumen, or any similar formation (internal aspiration lumen) 208 within it. In an embodiment, internal aspiration lumen 208 may have a diameter of 100 um or more (in an example 100-700 um, in an example 200-500 um, in an example 600-1100 um), allowing the collection of oocytes 160, as described below. Internal aspiration lumen 208 may be in fluid connection with a retrieval catheter aspiration opening 209 (as seen in FIG. 7A), or a plurality of retrieval catheter aspiration openings 209 (as seen in FIG. 7B), at its distal part, close to it, or separated from retrieval catheter tip by few centimeter (in an example 1-5 cm or 5-10 cm).

Retrieval catheter aspiration openings 209 may be located on retrieval catheter tip 213 (as seen in FIG. 7A), or on the circumference of retrieval catheter (as seen in FIG. 7B or FIG. 8A or 12A below for some examples). In the following example a single retrieval catheter aspiration opening 209 is described but the generalization to multiple retrieval catheter aspiration openings is implicit. Retrieval catheter aspiration openings 209 may allow the entrance and exit of other materials into internal aspiration lumen 208, as described below. In some cases, the edges of retrieval catheter aspiration opening 209 may be champfered, rounded or the like, to have smoothround entrance for biological material and oocytes, as described below.

FIG. 2C shows device 200 inside uterus 153. In an embodiment, introductory catheter 202 has a shape of a strait rigid body (e.g., cylinder, box or the like). FIG. 2D shows a tilt operation marked with arrow 261. Tilt operation 261 allows delivering the introductory catheter tip 210 close to the fallopian tube isthmus 159 (step 302 above). FIG. 2E shows another embodiment of device 200. In an embodiment, introductory catheter 202 includes an introductory catheter center part 212 having a shape of a strait rigid body (e.g., a cylinder, a cone, a box, etc.). In an embodiment, introductory catheter 202 further includes an introductory catheter front part 214. In an embodiment introductory catheter front part 214 has an arcuate (arched, bent) shape, such that the arcuate shape allows getting introductory catheter tip 210 in the proximity of fallopian tube isthmus 159.

FIG. 2E also shows a rotation operation 263 of introductory catheter 202 relative to the orientation shown in FIG. 2E. Rotation operation 263 allows switching the position of introductory catheter tip from being near one fallopian tubes isthmus 159 to the second fallopian tube isthmus 159.

FIG. 2F shows yet another embodiment of device 200. In an embodiment, the introductory catheter 202 includes an introductory catheter center rigid part 212, an introductory catheter front rigid part 216, and a bendable part 218. Bendable part 218 may be a hinge, a flexible hinge, may have an accordion (bellows) structure, etc. A bending operation 215 may be applied on bendable part 218, e.g., using an electrical motor, by pulling or pushing a control cord inside introductory catheter 202 (not illustrated), etc. By bending bendable part 218, introductory catheter tip 210 may be delivered closer to the fallopian tube isthmus 159.

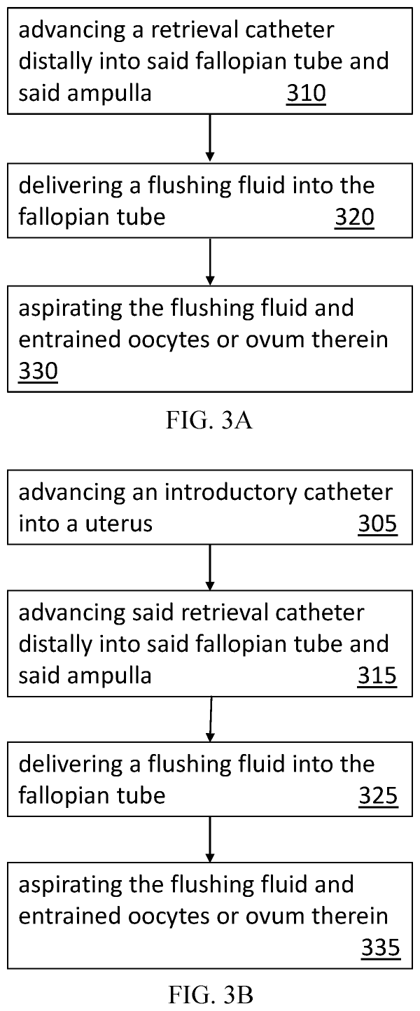

Reference is now made to FIG. 3A which is a flowchart of a method for oocytes or ovum retrieval, according to some embodiments of the present invention. In some embodiments, the method may be performed by providing device 200 and/or any device according to embodiments of the invention (e.g., devices 800, 1000, 1100, etc.). In step 310, a retrieval catheter may be advanced distally into the fallopian tube and the ampulla. For example, retrieval catheter 204 (or other retrieval catheters listed in this disclosure, e.g. 8204, 7204, etc.) may be advanced distally into fallopian tube 154 and ampulla 158. In some embodiments, retrieval catheter 204 may be pushed into retrieval position 252 inside fallopian tube 154, for example, until ampulla 158.

In some embodiments, prior to advancing retrieval catheter 204 into fallopian tube 154, introductory catheter 202 may be advanced into uterus 153 from the cervix 152, while retrieval catheter 204 is at initial position 251. In some embodiments, the method may include delivering introductory catheter tip 210 close to the fallopian tube isthmus 159. In step 320, a flushing fluid may be delivered through distal fluid delivery port 808 into the fallopian tube. For example, the flushing fluid may include for example a saline solution, saline with heparin, flushing medium, oocyte retrieval medium, oocyte culture medium, cell culture medium, etc. to be delivered via fluid delivery port 808 into the fallopian tube 154 and ampulla 158.

In step 330, the flushing fluid and entrained oocytes or ovum therein may be aspirated through at least one retrieval catheter aspiration opening 209. In some embodiments, oocytes or ovum swept in the flushing fluid may be retrieve from fallopian tube 154.

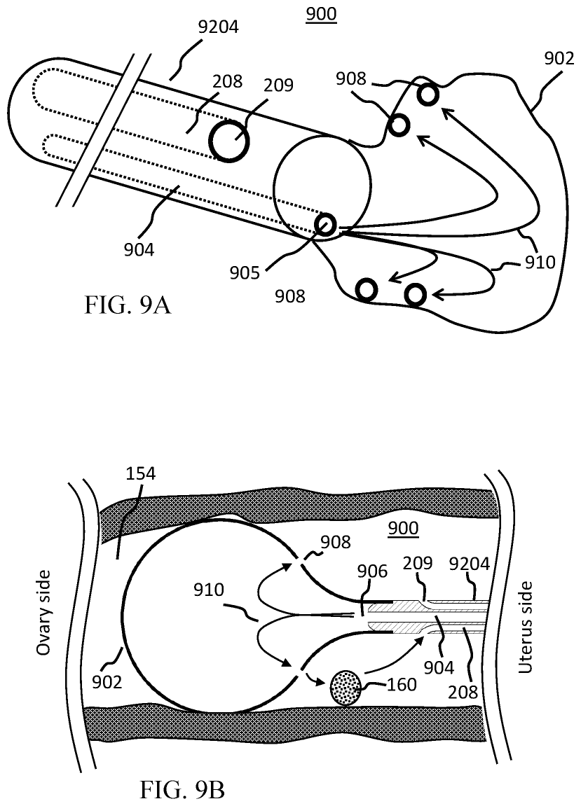

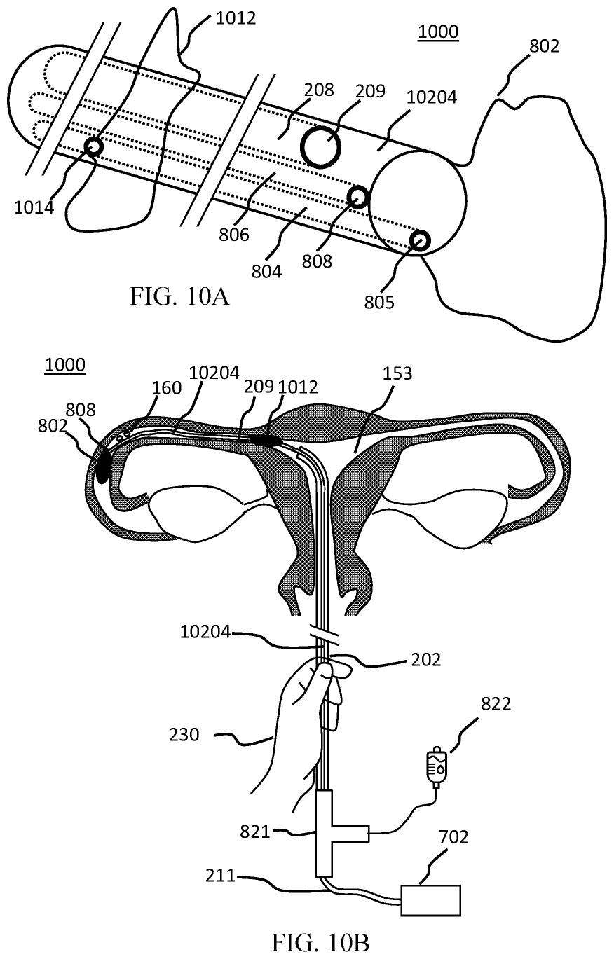

In some embodiments, the method may further include occluding a passage of the fluid, distally from fluid delivery port 808, by inserting a first occluding element 802 (illustrated and discussed with respect to FIGS. 8B and 10B), included in device 200, for deployment of a first occlusion point in fallopian tube 154. In some embodiments, other occluding elements may be used, for example, elements 902, 1901 and 2401 illustrated and discussed in FIGS. 9, 19 and 24.

In some embodiments, the method may further include occluding the passage of the fluid, proximity to fluid delivery port 808, by inserting a second occluding element 1012 (illustrated and discussed with respect to FIG. 10B), included in the device, for deployment of a second occlusion point in the fallopian tube.

In some embodiments, the method may be terminated when device 200 may be pulled from female reproductive system 150.

Reference is now made to FIG. 3B which is a flowchart of a method for oocytes or ovum retrieval, according to some embodiments of the present invention. In some embodiments, the method may be performed by providing device 200 and/or any device according to embodiments of the invention (e.g., devices 1100 or 2400).

In step 305, advancing an introductory catheter into a uterus, for example, introductory catheter 202 may be advanced into uterus 153. A dilator and\or an obturator device (both are not shown in any figure) may be used to facilitate the insertion of introductory catheter 202 into uterus 153. In step 315, a retrieval catheter may be advanced distally into the fallopian tube and the ampulla. For example, retrieval catheter 204 may be advanced distally into fallopian tube 154 and ampulla 158.

In step 315, a retrieval catheter may be advanced distally into the fallopian tube and the ampulla. For example, retrieval catheter 204 may be advanced distally into fallopian tube 154 and ampulla 158. In step 325, a flushing fluid may be delivered through distal fluid delivery port 808 into the fallopian tube. For example, the flushing fluid may include for example a saline solution, saline with heparin, flushing medium, oocyte retrieval medium, oocyte culture medium, cell culture medium, etc. to be delivered via fluid delivery port 808 into the fallopian tube 154 and ampulla 158.

In step 335, the flushing fluid and entrained oocytes or ovum therein may be aspirated through at least one retrieval catheter aspiration opening 209.

In some embodiments, the method may further include occluding a passage of the fluid, distally from fluid delivery port 808, by inserting a first occluding element 802 (illustrated and discussed with respect to FIGS. 8B and 10B), included in device 200, for deployment of a first occlusion point in fallopian tube 154.

In some embodiments, the method may further include occluding the passage of the fluid, proximity to fluid delivery port 808, by inserting a second occluding element 1012 (illustrated and discussed with respect to FIG. 10B), included in the device, for deployment of a second occlusion point in the fallopian tube.

In some embodiments, the methods of FIGS. 3A and 3B may be terminated when device 200 may be pulled from female reproductive system 150.

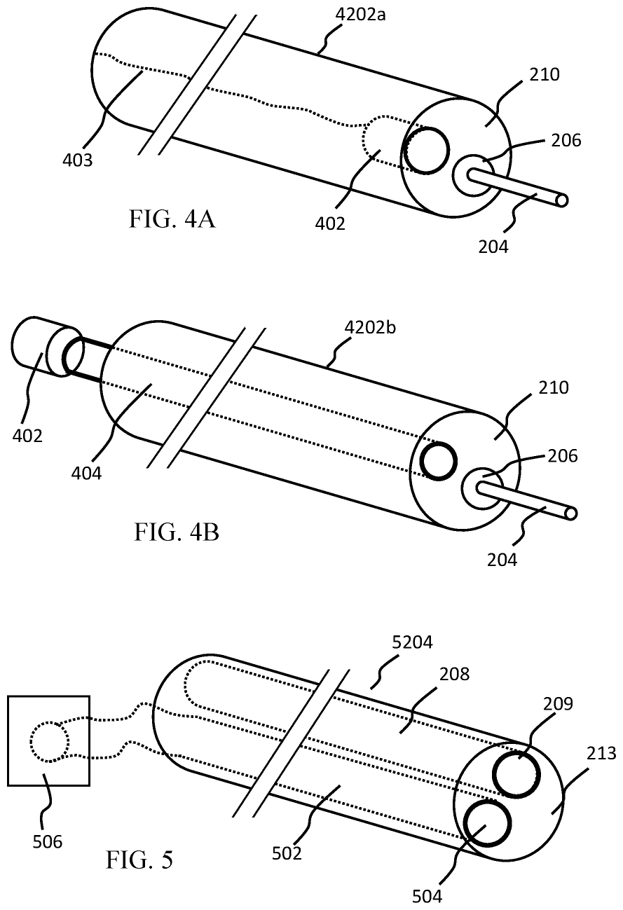

In some embodiments, the methods of FIGS. 3A and 3B may further include a use of a camera (e.g., camera 402 illustrated and discussed with respect to FIG. 4A) to guide retrieval catheter 204 and/or introductory catheter 202.

In some embodiments, the methods of FIGS. 3A and 3B may further include guiding retrieval catheter 204 and/or introductory catheter 202 using external imaging system. For example, the delivery of retrieval catheter 204 to retrieval position may be guided using optical fiber 502 (illustrated and discussed with respect to FIG. 5) embedded in retrieval catheter 204 and included in an external imaging system.

In some embodiments, the methods of FIGS. 3A and 3B may further include using a guide wire (e.g., “Seldinger” wire, not illustrated) for guiding retrieval catheter 204 to the retrieval position. For example, the guide wire may be inserted into introduction catheter lumen 206 and may further be pushed to introductory catheter tip 210. Additionally, the guide wire may be inserted into fallopian tube 154 to be pushed towards ampulla 158. In some embodiments, retrieval catheter 204 may slide over the guide wire.

In some embodiments, the methods of FIGS. 3A and 3B may be repeated with respect to any one of the fallopian tubes. In some cases, the delivery of introductory catheter tip 210 close to the fallopian tube isthmus 159 may be guided using a camera 402 as, shown in some examples in FIGS. 4A-4B.

FIG. 4A shows one example of modified introductory catheter 4202a which is similar to introductory catheter 202 with the addition of camera 402 manifested as miniature digital camera located on introductory catheter tip 210. In one example, camera 402 may be a complementary metal oxide semiconductor (CMOS) or a charged-coupled-device (CCD) camera, with a miniature lens, allowing, e.g., a 50-180 degrees field of view (FOV), capturing color images or monochrome (black-and-white) images. Camera 402 may image in the visible and/or near infrared (IR) spectrum. In another example, camera 402 may be manifested as a bundle of optical fibers transmitting images from the tip of introductory catheter 202 to the introductory catheter back part 207 where an image sensor is located. In one case, camera 402 may be equipped with illumination source such as a light emitting diode (LED). In one case, camera 402 may be accompanied by optical fiber passing along modified introductory catheter 4202a and delivering illumination from outside of the female reproductive system, thus serving as illumination source for camera 402. Electrical wiring 403 may pass along modified introductory catheter 4202a and outside of the female reproductive system (not shown), where it is connected to a driving circuitry (not shown).

Electrical wiring 403 may transmit electrical signals between camera 402 and driving circuitry. FIG. 4B shows another example of modified introductory catheter 4202b which is similar to introductory catheter 202 with the addition of camera 402 manifested as a digital camera located outside of the female reproductive system and near the rear part of introductory catheter 4202b. Telescopic imaging system 404 is embedded inside modified introductory catheter 4202. Telescopic imaging system 404 is used to deliver images from within the uterus to camera 402. Further detailed design considerations of telescopic imaging system are known in the art. Camera 402 may be of other formats, which are not illustrated, for example a fiber optics camera or any similar camera which may deliver images from within the uterus and show a clear view of the fallopian tube isthmus 159. In case of using camera 402, an optional step may be added in the description of FIGS. 3A-3B above, wherein camera 402 may be used to guide introductory catheter 202 (or 4202a or 4202b).

In some cases, the delivery of introductory catheter tip 210 close to the fallopian tube isthmus 159 may be guided using an external imaging system (external relative to device 200), such as abdominal or vaginal sonar imaging system (also known as ultrasound imaging system, and abbreviated hereafter simply as US), magnetic resonance imaging (MM) system, X-ray imaging system or a like. External imaging system is not seen in figures. In case of using an external imaging system, an optional step may be added in the description of FIGS. 3A-3B above, wherein an external imaging system may be used to guide introductory catheter 202.

In some cases, the delivery of retrieval catheter 204 to a retrieval position may be guided using a camera 500 embedded in retrieval catheter 204 to image the interior of fallopian tube 154. FIG. 5 shows modified retrieval catheter 5204. Modified retrieval catheter 5204 is similar to retrieval catheter 204 with the following additions: an optical fiber 502 is embedded in modified retrieval catheter 5204 alongside to internal aspiration lumen 208. A lens 504 is positioned at the tip of optical fiber 502. Lens 504 may be an imaging lens, made for example from glass or plastic. Lens 504 is designed to image the internal fallopian tube into the edge of optical fiber 502.

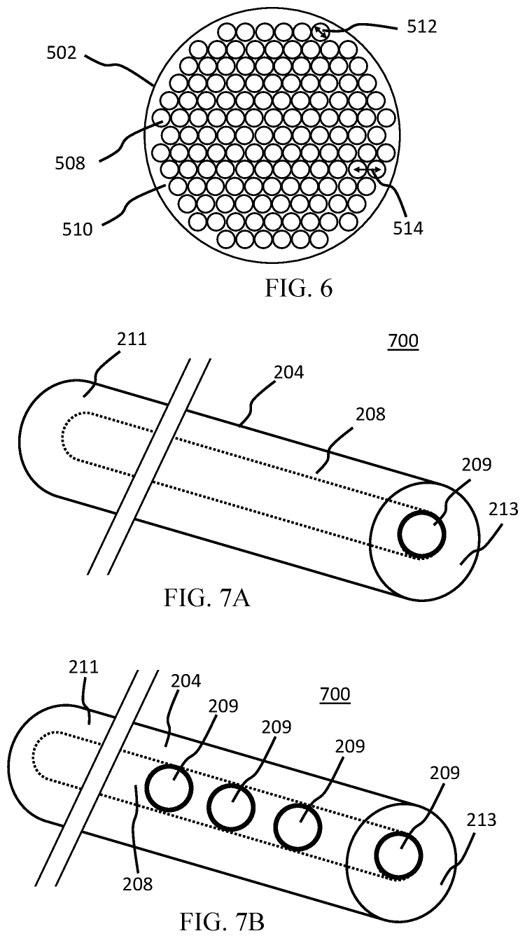

FIG. 6 shows a cross section of optical fiber 502. Optical fiber 502 may be a multicore optical fiber comprising plurality of cores 508. In one example multicore fiber is made of plastic, with cores 508 made from Polystyrene and cladding 510 made from Silicone. In one example, optical fiber 502 may be made of glass. Optical fiber 502 may have, for example, few hundreds to few tens of thousands of cores. In some cases, each core 508 has a diameter 512 of less than a micrometer (microns or um) e.g., 0.1-0.9 um. In some cases, each core 508 has a diameter 512 of few micrometers (e.g., 1-15 um). In some cases, the distance between each two adjacent cores 514 may be for example few um (e.g., 1-10 um). In some cases, the distance between each two adjacent cores 514 may be for example more than 10 um (e.g., 10-40 um). With reference back to FIG. 5, optical fiber 502 may continue outside of the female reproductive system and deliver an image to an image sensor 506, for example a CMOS of CCD image sensor. In some cases, camera 500 may be accompanied by optical fiber passing along modified retrieval catheter 5204 and delivering illumination from outside of female reproductive system, thus serving as illumination source for camera 500. In case of using camera 500, a wherein camera 502 may be used to image the interior of fallopian tube 154 and to guide retrieval catheter 204.

In some cases, the delivery of retrieval catheter 204 to retrieval position may be guided using an external imaging system (external relative to device 200) such as abdominal US, vaginal US, MRI system, X-ray imaging system or a like. External imaging system is not seen in the figures. In case of using an external imaging system, an optional step may be added in the description of FIGS. 3A-3B, wherein an external imaging system may be to guide retrieval catheter 204.

In some cases, the delivery of retrieval catheter to retrieval position may be guided using an optical coherence tomography (OCT) imaging system positioned at a catheter tip and mounted on retrieval catheter 204. OCT tip pulled along ampulla 158 may create an image of the internal surface of the fallopian tube 154 and locate oocytes 160. Oocytes 160 may be counted to match with the number of oocytes retrieved later. In case of using OCT tip, an optional step may be added in the description of FIGS. 3A-3B, wherein an OCT tip may be to guide retrieval catheter 204.

In an embodiment, device 200 may include a guide wire (not shown), e.g., a Seldinger wire, 0.014″ guide wire, 0.038″ guide wire, etc. In an embodiment, the delivery of retrieval catheter to retrieval position may be guided using the guide wire. In an example guide wire may be semi-rigid wire made from a metallic material. The guide wire is designed to enter fallopian tube 154 and follow the shape of fallopian tube 154. The guide wire is designed to have an external diameter smaller than the diameter of internal aspiration lumen 208 (e.g., in the range of 100-300 um). In case of using an external imaging system an optional step be added in the description of FIGS. 3A-3B, comprising inserting a guide wire into introductory catheter lumen 206, pushing the guide wire to introductory catheter tip 210, inserting the guide wire into fallopian tube 154, pushing the guide wire to fallopian tube ampulla 158, and sliding retrieval catheter 204 over the guide wire into fallopian tube 154.

FIGS. 7A-C show exemplary oocytes retrieval device 700. Device 700 may be included in device 200 described above. In some embodiments, device 700 is designed to enter female reproductive system in transcervical and\or transvaginal procedure.

FIG. 7A shows an embodiment of exemplary device 700, which includes retrieval catheter 204. Retrieval catheter 204 includes internal aspiration lumen 208. Retrieval catheter 204 further includes one or more retrieval catheter aspiration openings 209. Retrieval catheter aspiration openings 209 are located at the distal part of retrieval catheter 204 (e.g., at retrieval catheter tip 213). Retrieval catheter 204 further includes retrieval catheter back part 211.

FIG. 7B shows another embodiment of exemplary device 700, where retrieval catheter 204 includes a plurality of retrieval catheter aspiration openings 209 along a sidewall of retrieval catheter 204 (e.g., 2-5 openings, 2-10 openings, 5-15 openings). The plurality of retrieval catheter aspiration openings 209 may be distributed along one side or region of a sidewall of retrieval catheter 204, and\or about the entire sidewall of retrieval catheter 204.

FIG. 7C shows device 200 in operation with respect to female reproductive system 150. As can be seen, retrieval catheter 204 is located at a retrieval position inside fallopian tube 154. In FIG. 7C, retrieval catheter aspiration openings 209 is located in the ampulla 158, and close to oocytes 160 (e.g., within 1-5 centimeters distal or proximal to oocytes 160). According to an example, suctioning may be operated in internal aspiration lumen 208 to retrieve oocytes into internal aspiration lumen 208 in retrieval catheter 204. In FIG. 7C, retrieval catheter back part 211 is shown to continue outside of the female reproductive system, out of introductory catheter back part 207 and into pump 702. Pump 702 may create the suctioning operation. Pump 702 may be, e.g., a mechanical pump, a syringe, electrically controlled pump etc., used to create a suction force in internal aspiration lumen 208. According to an example, pump 702 may operate at suction pressure of 0.1-0.9 atmospheres. In one example (not shown), introductory catheter back part 207 may be connected to a test tube from one port and pump 702 may be connected to the same test tube from another port, such that oocytes suctioned from fallopian tube 154 may be collected in the test tube. The total length of retrieval catheter 204 from retrieval catheter tip 213 to retrieval catheter back part 211 may be 10-20 cm, or 20-40 cm, or 40-100 cm, or more than 100 cm.

FIGS. 8A-8B show another example of oocyte retrieval device 800. In some embodiments, device 800 is designed to enter female reproductive system in transcervical and\or transvaginal procedure. Device 800 includes a modified retrieval catheter 8240. Device 800 further includes an occluding element 802. In one example occluding element may be manifested as balloon 802 as seen in FIG. 8B. In another example, occluding element may be manifested as stent covered with membrane, as exampled in FIG. 19B, below. The following description detail the use of balloon as an occluding element, with possible changes to other type of occluding elements are obvious. Balloon 802 is located near a distal part of retrieval catheter 8204 (e.g., just before or just after retrieval catheter tip 213). When inside the female reproductive system and inflated, balloon 802 may occlude fallopian tube 154 such that liquids cannot pass from one side of balloon 802 to its other side. Balloon 802 may be made for example from urethane, latex, polyethylene terephthalate (PET), nylon, or any suitable material.

FIG. 8A shows modified retrieval catheter 8204 distal end and balloon 802. Modified retrieval catheter 8204 is similar to retrieval catheter 204 with the following additions: Modified retrieval catheter 8204 further includes an inflation lumen 804, alongside to internal aspiration lumen 208. Inflation lumen 804 allows the inflation of balloon 802 with any suitable fluid, e.g., liquid (e.g., saline solution, oocyte medium) or gas (e.g., air, oxygen, nitrogen, CO2, etc.) via inflation openings 805. Modified retrieval catheter 8204 further includes fluid delivery lumen 806. Fluid delivery lumen 806 may allow discharge of liquid into the fallopian tube 154. In one example, in modified retrieval catheter 8204, retrieval catheter aspiration openings 209 is set proximal to the tip of retrieval catheter by few mm (10-100 mm).

FIG. 8B shows device 820 used in female reproductive system. Device 820 include device 800. According to one example shown in FIG. 8B, Device 820 further include introductory catheter 202. According to one example (not shown) device 820 does not include an introductory catheter. Device 820 further include fitting 821, fitting 821 is connected to retrieval catheter back part 211; fitting 821 allows connection to the three retrieval catheter lumens (208, 804 and 806). Device 820 further include a pump 702, pump 702 is connected to retrieval catheter lumen using fitting 821. Pump 702 may create suction force in internal aspiration lumen 208. Pump 702 may be for example mechanical pump, a syringe, electrically controlled pump etc. Device 820 further includes a means to inflate balloon 802. Such means can be a syringe 823 (as shown in FIG. 8B), a pump, etc. Syringe 823 is connected to a second retrieval catheter lumen using fitting 821. Syringe 823 may inject any fluid or gas or liquid into the second internal lumen and into balloon 802. Device 820 may further include means to provide liquid connected to fluid delivery lumen 806 using fitting 821. Means to provide liquid may be an infusion bag 822 (as seen in FIG. 8B), a syringe, a pump. etc. According to an example, means to provide liquid may deliver liquid at a pressure of 0.1-1 atmospheres or at a pressure of 1-5 atmospheres or at a pressure of 3-15 atmospheres.

According to an example, a method of the present disclosure may comprise the following steps:

(i) Inserting modified retrieval catheter 8204 into fallopian tube 154 while balloon 802 is not inflated;
(ii) positioning balloon 802 distal to the ampulla 158 (e.g., in the infundibulum 157);
(iii) inflating balloon 802 via liquid or gas from inflation lumen 804, e.g., as shown in FIG. 8B;
(iv) providing liquid (e.g., saline solution, oocyte medium) from fluid delivery lumen 806 to flush oocytes toward retrieval catheter aspiration openings 209 (proximal direction); and
(v) collecting oocytes into internal aspiration lumen 208 using suctioning operation, such that internal aspiration lumen 208.

Inflation of balloon 802 may be done for example using a syringe 823 connected to second retrieval catheter lumen or by other means known in the art. Liquid used to flush oocytes may be sourced for example from infusion bag 822, from a syringe or from any other liquid source connected to fluid delivery lumen 806. Suctioning operation may be done for example using pump 702.

According to one example, the following method may be used for positioning the balloon to block the fallopian tube (steps (ii) and (iii): the balloon may be first positioned distal to ampulla 158 and inflated such that is doe not fully block the fallopian tube. The balloon may then be pulled back (proximally) until resistance is sensed by the operator (e.g. medical doctor) and the balloon in wedged into the fallopian tube. This method applies for all balloon embodiments above and below.

FIGS. 9A-9B show another example of oocyte retrieval devices 900 and 920. FIG. 9A shows Device 900; Device 900 includes modified retrieval catheter 9204 and balloon 902. FIG. 9A shows modified retrieval catheter 9204 distal end. Modified retrieval catheter 9204 is similar to retrieval catheter 204 with the following additions: Modified retrieval catheter 9204 includes a fluid delivery lumen 904 and optionally internal aspiration lumen 208. Fluid delivery lumen 904 is connected to balloon 902 via inflation opening 905. Fluid delivery lumen 904 allows the inflation of balloon 902 with liquid (e.g., saline solution, oocyte medium) provided from inflation opening 905. Balloon 902 is located at the distal end of modified catheter 9204, e.g., near retrieval catheter tip 213. When inflated, balloon 902 may occlude fallopian tube 154 such that liquids cannot pass from one side of balloon 902 to the other side. Balloon 902 further includes exit holes 908, which allow access liquid to discharge into fallopian tubes 154 and flush oocytes toward retrieval catheter aspiration openings 209, shown, e.g., in FIGS. 7A-7B. In FIG. 9A there are four exit holes 908, but there can be any number of exit holes (for example two in FIG. 9B). Arrows 910 in FIG. 9B show the propagation direction of liquid: from inflation opening 905 into balloon 902, then into exit holes 908, then into the fallopian tube, and then toward retrieval catheter aspiration openings 209. According to one example, exit holes 908 may be formed as breach opening, similar to breach opening 1408 described below. According to such example, exit holes 908 may be initially sealed to the exit of fluid from balloon 902, but when liquid pressure in balloon 902 exceed a threshold value (e.g., a threshold value in the range of 1-3 atmospheres), breach may occur and exit holes my open for the spill of liquid. According to one example exit hole 908 may be covered by a break line which breaks above threshold pressure (similar to embodiment 1410). According to one example exit hole 908 may be covered by a membrane which moves aside above threshold pressure (similar to embodiment 1422).

Device 920 may be used within a female reproductive system. Device 920 includes device 900, introductory catheter 202, fitting 921, pump 702 and liquid source 822. Fitting 921 is connected to retrieval catheter back part 211 and allows connection to the two fluid delivery lumens 806 and 904. In an embodiment, liquid source 822 may deliver liquid via fitting 922 and into fluid delivery lumen 904. Liquid source 822 may be for example a syringe, an infusion bag, a pump, etc. Introductory catheter 202 is positioned in uterus 153, retrieval catheter 9204 is positioned in fallopian tube 154, balloon 902 is inflated and located behind oocytes 160. Liquid may be delivered from fluid delivery lumen 904 trough balloon 902 and trough exit holes 908 to fallopian tube 154. Liquid may flow toward retrieval catheter aspiration openings 209 with oocytes 160.

In FIG. 9B, balloon 902 is inflated and is occluding fallopian tube 154. Arrows 910 show the propagation direction of liquid, as described above. After liquid arrives close to retrieval catheter aspiration openings 209, liquid is pulled into internal aspiration lumen 208 by suction force created by pump 702.

According to one example, device 900 allows the inflation of the balloon 902 without spilling of flushing liquid into fallopian tube 154, and device 900 further allows the spilling of flushing liquid into fallopian tube 154 without deflating balloon 902.

According to an example the following method may be used to retrieve oocytes using modified retrieval catheter 9204:

(i) Inserting modified retrieval catheter 9204 into fallopian tube 154 while balloon 902 is not inflated;
(ii) positioning balloon 902 distal to ampulla 158 (e.g., in the infundibulum 157);
(iii) inflating balloon 902, e.g., as shown in FIG. 9A, using liquid (e.g., saline solution, oocyte medium, etc.);
(iv) continue flushing liquid into balloon 902 until liquid is discharge from exit holes 908 to fallopian tube 154; liquid will flush oocytes toward retrieval catheter aspiration openings 209; and
(v) collecting oocytes into internal aspiration lumen 208 using suction operation.

FIGS. 10A-10B show another example of oocyte retrieval device 1000. device 1000 includes retrieval catheter 10204. FIG. 10A shows a modified retrieval catheter 10204 distal end. Device 1000 is similar to device 800 and modified retrieval catheter 10204 is similar to modified retrieval catheter 8204 with the following additions: device 1000 includes a second balloon 1012. Balloon 1012 is connected to inflation lumen 804 by second retrieval catheter lumen back opening 1014; Balloon 1012 may be inflated alongside balloon 802. Balloon 1012 is located few mm (e.g., 5-100 mm) proximally to both retrieval catheter aspiration openings 209 and fluid delivery port 808. When inflated, balloon 1012 may occlude fallopian tube 154 such that liquids cannot pass from one side of balloon 1012 to the other side. According to an example, the following method of the present disclosure may be used in conjunction with modified retrieval catheter 10204:

(i) Inserting modified retrieval catheter 10204 into fallopian tube 154 while balloons 802 and 1012 are not inflated;
(ii) positioning balloon 802 behind ampulla 158 (e.g., in infundibulum 157);
(iii) positioning balloon 1012 before ampulla 158 (e.g., in isthmus 159);
(iv) inflating balloons 802 and 1012, as shown in FIG. 10B;
(v) using liquid (e.g., saline solution, oocyte medium) from fluid delivery lumen 806 to flush oocytes toward retrieval catheter aspiration openings 209; and (vi) collecting oocytes into internal aspiration lumen 208 using suction operation.

This method may be assisted by internal cameras such as 402 or 502 described above or by external imaging system (e.g. US, X-ray etc.).

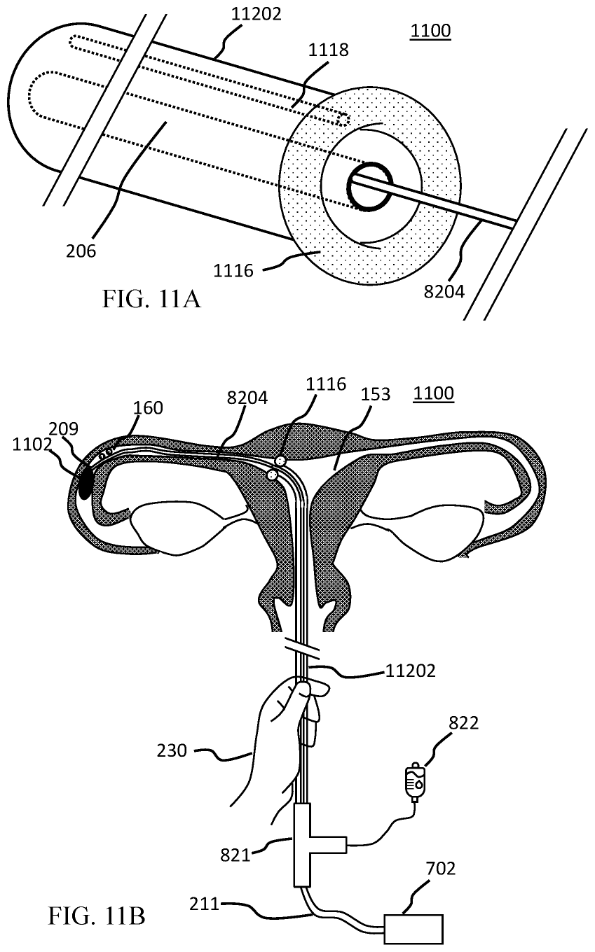

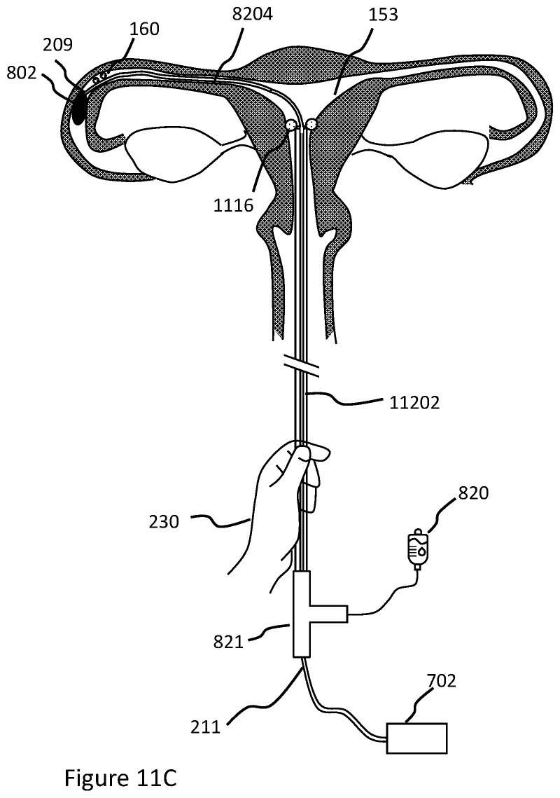

FIGS. 11A-11B show another example of retrieval device 1100. Device 1100 includes modified introductory catheter 11202. Device 1100 further include a modified retrieval catheter 11204. In an example, modified retrieval catheter 11204 may be any of retrieval catheters shown above of below (e.g., 204, 8204, 9204, 12204 etc.). In one example, modified retrieval catheter 11204 may be irrigation catheter 19206 described below. Device 1100 further include an occluding element 1102, for example balloon 802, occluding element 1901 etc. FIG. 11A shows modified introductory catheter 11202 distal part along with a section of retrieval catheter 8204 as an example. FIG. 11B shows modified introductory catheter 11202 integrated with modified retrieval catheter 8204. device 1100 is similar to device 800 and modified introductory catheter 11202 is similar to introductory catheter 202 with the following additions: modified catheter 11202 includes funneling element 1116 which is a means for funneling liquids coming out of fallopian tube 154 toward the uterus side. In an example shown in FIG. 11A, funneling element 1116 is demonstrated as balloon 1116 which has a torus shape when inflated. In other examples, funneling element 1116 may be other means for funneling liquids coming out of fallopian tube 154 toward the uterus side, e.g., rubber funnel tip, spring funnel, etc. Modified introductory catheter 11202 may further include an inflation lumen 1118. In an embodiment, inflation lumen 1118 allows the inflation of balloon 1116 with liquid (e.g., saline solution, oocyte medium) or gas (e.g., air, oxygen, nitrogen, CO2, etc.).

FIG. 11B shows device 1100 with modified introductory catheter 11202 and modified retrieval catheter 8204, occluding element 1102, and female reproductive system. FIG. 11B shows modified introductory catheter 11202 inside uterus 153, funneling element 1116 is and funneling any liquid coming out of fallopian tube 154, modified retrieval catheter 8204 located at retrieval position inside fallopian tube 154, and balloon 802 inflated in fallopian tube 154 behind oocytes 160. FIG. 11B further shows liquid source 822 connected to retrieval catheter back part 211; liquid source 822 can send liquid through fitting 821 to fluid delivery lumen 806, and pump 702, which can operate suction through fitting 821 on introductory catheter lumen 1118. In FIG. 11B, funneling element 1116 is positioned in close to isthmus 159, such that fluids coming out of fallopian tube 154 are blocked from entering uterus 153.

FIG. 11C shows device 1100, with the modification that funneling element 1116 is positioned in uterus 153 and close to the cervix 152; in such a configuration, fluids coming out of fallopian tube 154 may go through uterus 153 before being pumped out by introductory catheter 11202.

According to an example, the following method may be used to retrieve oocytes using modified introductory catheter 11202 and modified retrieval catheter 8204:

(i) Inserting modified introductory catheter 11202 into the uterus 153 from the cervix 152, while retrieval catheter 204 is at initial position 251 and balloon 1116 is not inflated;
(ii) delivering introductory catheter tip 210 close to the fallopian tube isthmus 159;
(iii) inserting modified retrieval catheter 11204 into fallopian tube 154;
(iv) pushing modified retrieval catheter 11204 to retrieval position 252 inside fallopian tube 154. In an example, modified retrieval catheter 204 is pushed beyond the ampulla 158;
(v) positioning occluding element 802 behind ampulla 158 (e.g., in the infundibulum 157);
(vi) occluding fallopian tube 154 with occluding element 1108, e.g., as exampled in FIG. 11B;
(vii) using liquid (e.g., saline solution, oocyte medium) to flush oocytes toward uterus 153;
(viii) using introductory catheter 11202 and funneling element 1116 to funnel liquid into introductory catheter lumen 206; and
(ix) collecting oocytes into introductory catheter lumen 206 using suction operation.

This method may be assisted by internal cameras such as 402 or 502 described above or by external imaging system (e.g., US, Xray etc.).

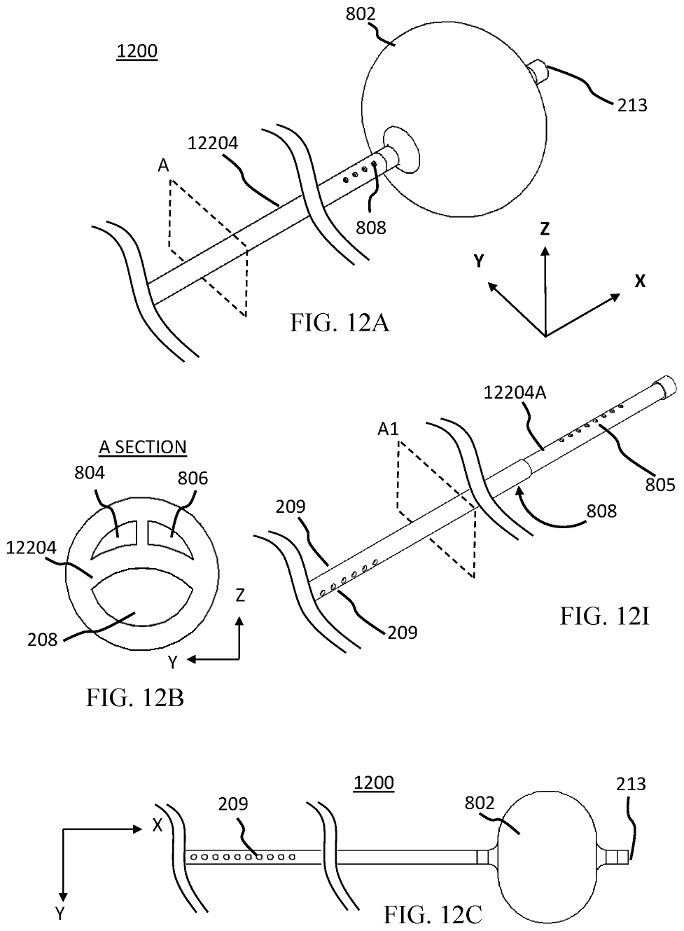

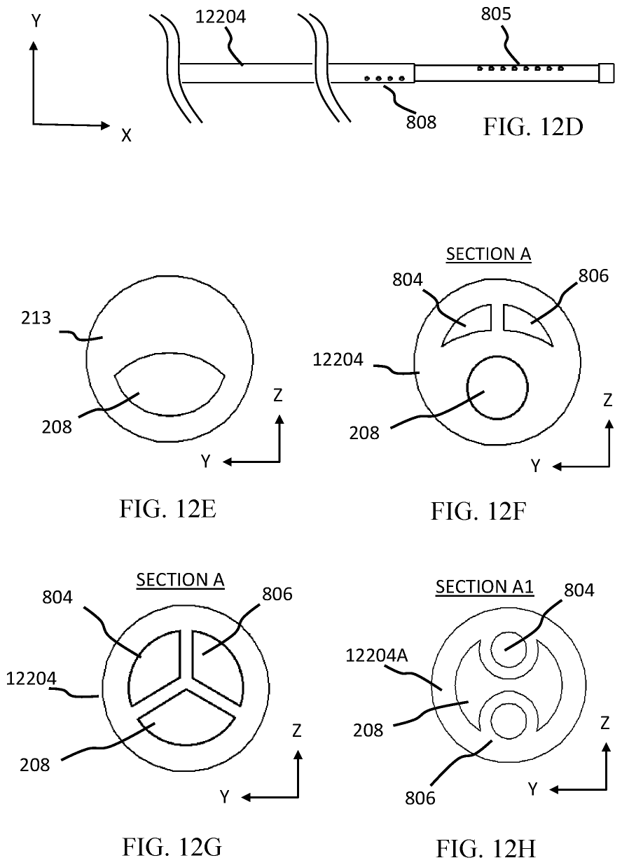

FIGS. 12A-I show another example of retrieval device 1200. FIGS. 12A-I show coordinate system x-y-z for illustrative purposes, which is provided for clarity purposes only, and does not reflect on device operation.

FIG. 12A is an isometric projection. Device 1200 includes a modified retrieval catheter 12204. Modified retrieval catheter 12204 includes parts similar in functionality and numbering to modified retrieval catheter 8204. Modified retrieval catheter 12204 may be made from any flexible biocompatible material, such as: silicone, polyurethane, Teflon, PTFE, FEP, etc. In an example, modified retrieval catheter 12204 may have a circular cross section with a diameter of, e.g., 0.8-1.4 mm or 1.2-1.6 mm, allowing it to pass in the isthmus 159 of female reproductive system.

FIG. 12B shows a cross section of modified retrieval catheter 12204 along plane A marked in FIG. 12A. Modified retrieval catheter 12204 includes an aspiration lumen 208, an inflation lumen 804, and a fluid delivery lumen 806.

FIG. 12C shows device 1200 and modified retrieval catheter 12204 from a bottom view. Modified retrieval catheter 12204 further includes Retrieval catheter aspiration openings 209. Retrieval catheter aspiration openings 209 are in fluid connection with internal aspiration lumen 208; retrieval catheter aspiration openings 209 thus allow suctioning into internal aspiration lumen 208 of, e.g., oocytes and/or ovum and/or other biological material (epithelial cells, ovarian cells, etc.). In FIG. 12C, eight retrieval catheter aspiration openings 209 are marked as an example, but any number of retrieval catheter aspiration openings 209 may be used (e.g., 1-50). In an example, retrieval catheter aspiration openings 209 are located about a sidewall of modified retrieval catheter 12204 proximally to modified retrieval catheter tip 213 by a few centimeters (e.g., 4-8 cm); when positioned inside fallopian tube 154, retrieval catheter aspiration openings 209 are proximal to the ampulla (toward the uterus side) while modified retrieval catheter tip 213 is distal to the ampulla (e.g., towards the ovaries).

With reference back to FIG. 12A, modified retrieval catheter 12204 includes fluid delivery ports 808. fluid delivery ports 808 are in fluid connection with a fluid delivery lumen 806. fluid delivery ports 808 allow a discharge of liquids from fluid delivery lumen 806 to the fallopian tube 154. Now in FIGS. 12A and 12C, device 1200 further includes balloon 802. Balloon 802 is sealed (glued, connected) to the distal part of modified retrieval catheter 12204. In FIGS. 12A and 12C balloon 802 is shown in an inflated form, where it has a shape having a radial symmetry around x axis and a cross section (along Y-Z plane) of a circle having a diameter of at least 10 mm, or at least 15 mm or at least 20 mm; balloon 802 is designed to occlude the distal parts of fallopian tube 154 (e.g., ampulla 158 or infundibulum 157). In the non-inflated form (not shown), balloon 802 may be flat with the dimensions of modified retrieval catheter 12204, such that the cross section around the balloon area may be similar to the cross section along modified retrieval catheter 12204 (e.g. when not inflated, balloon 802 may have cross section dimensions not exceeding the dimensions of cross section of retrieval catheter 12204 by more than 0.2 mm). FIG. 12D shows modified retrieval catheter 12204 in a side perspective (balloon 802 is not shown). Modified retrieval catheter 12204 includes inflation openings 805. Inflation openings 805 are in fluid connection with inflation lumen 804. Balloon 802 may be inflated with gas or liquid via inflation lumen 804 and inflation openings 805.

Attention is drawn to FIGS. 12B and 12E. In an example, internal aspiration lumen 208 cross section has an incircle with a diameter of at least 0.3 mm or at least 0.4 mm or at least 0.5 mm. FIG. 12E shows one example of the face of retrieval catheter tip 213. In an example, internal aspiration lumen 208 may have an opening at retrieval catheter tip 213. Internal aspiration lumen 208 may allow the passage of a guidewire with a similar diameter as the incircle of internal aspiration lumen 208 cross section. In the example in FIG. 12E, inflation lumen 804 and fluid delivery lumen 806 are occluded at the tip of retrieval catheter 212. In another example embodiment (not shown) the tip of retrieval catheter lumen may be occluded to all three retrieval catheter cavities.

FIG. 12F show alternative cross section of modified retrieval catheter 12204, along cross section A in FIG. 12A. in an embodiment, fluid delivery lumen 208 has a circular cross section. In some cases, the circular cross section may be dimensioned to accommodate a guide wire used to introduce retrieval catheter into the fallopian tube 154. In some cases, the circular cross section may reduce the risk of an oocyte being trapped in internal aspiration lumen 208.

FIG. 12G shows another alternative cross section of modified retrieval catheter 12204, along cross section A in FIG. 12A. In an embodiment, the three lumens (208, 804, 806) have substantially equal cross-sectional shape and/or dimensions. In an example the wall thickness of each lumen presented in FIGS. 12A-H and in other embodiment in this disclosure may be in the range of 50-200 um.

FIGS. 12H-I show another alternative modified retrieval catheter 12204A similar in functionality and part numbers to modified retrieval catheter 12204, with different cross sections. FIG. 12I shows modified retrieval catheter 12204A in an isometric perspective, while FIG. 12H shows a cross section of modified retrieval catheter 12204A along plane A1 in FIG. 12I. Advantageously, the cross section of modified retrieval catheter 12204A allows retrieval catheter aspiration openings 209 to be positioned on two opposite sides of modified retrieval catheter 12204A (two opposing sides along its longitudinal Y dimension as marked in FIG. 12I, one side is not seen in the perspective view but marked only). Positioning retrieval catheter aspiration openings 209 on opposing sides of modified retrieval catheter 12204A increases the chance of an oocyte being pulled into internal aspiration lumen 208 by suction force. Inflation 805 and fluid delivery port 808 are located on two opposite sides along the Z dimension (fluid delivery ports 808 are not seen in the isometric perspective but their position is marked).

Device 1200 may be integrated within device 820. Device 1200 may be used for oocyte retrieval.

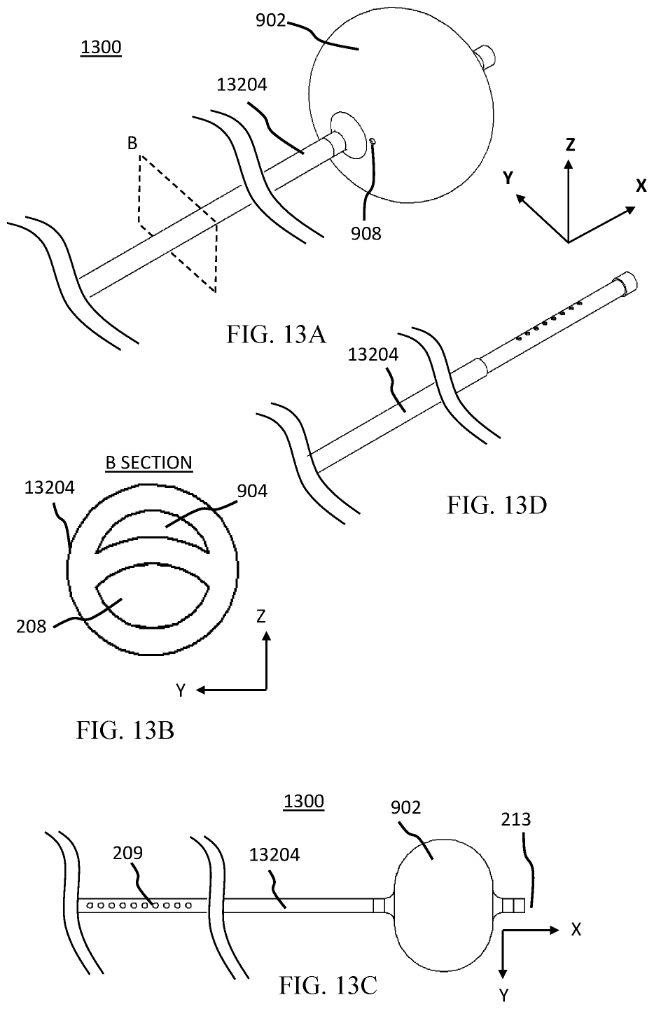

FIGS. 13A-D show another example of retrieval device 1300. As in FIGS. 12A-I, FIGS. 13A-D show coordinate system x-y-z for illustrative purposes only and does not reflect on the device operation. Device 1300 includes a modified retrieval catheter 13204. FIG. 13A device 1300 in an isometric projection. Device 1300 includes modified retrieval catheter 13204. Modified retrieval catheter 13204 includes parts similar in functionality and numbering to modified retrieval catheter 9204. Modified retrieval catheter 13204 may be made from a flexible biocompatible material, such as: silicone, polyurethane, Teflon etc. In an example, modified retrieval catheter 13204 may have a circular cross section with a diameter of 0.8-1.4 mm, allowing it to pass in the isthmus 159 of a female reproductive system. FIG. 13B shows a cross section of modified retrieval catheter 13204 along plane B marked in FIG. 13A. Modified retrieval catheter 13204 includes an internal aspiration lumen 208 and a fluid delivery lumen 904. In an example, internal aspiration lumen 208 cross-section has an incircle with a diameter of at least 0.3 mm or at least 0.4 mm or at least 0.5 mm; in an example, internal aspiration lumen 208 may be dimensioned to allow the passage of a guidewire with a similar diameter as the incircle of internal aspiration lumen 208 cross section.

FIG. 13C shows device 1300 and modified retrieval catheter 13204 from a bottom view. Modified retrieval catheter 13204 further includes retrieval catheter aspiration openings 209. Retrieval catheter aspiration openings 209 are in fluid connection with an internal aspiration lumen 208; retrieval catheter aspiration openings 209 allow suctioning of material, e.g. oocytes and/or ovum into internal aspiration lumen 208. In FIG. 12C, eight retrieval catheter aspiration openings 209 are marked as an example, but any number of retrieval catheter aspiration openings 209 may be used (e.g., 1-50). In an example, retrieval catheter aspiration openings 209 are located proximally to modified retrieval catheter tip 213 by few centimeters (e.g., 4-8 cm); when positioned inside fallopian tube 154, retrieval catheter aspiration openings 209 are proximal to the ampulla (toward the uterus side) while modified retrieval catheter tip 213 is distal to the ampulla (toward the ovaries).

Now in FIGS. 13A and 13C, device 1300 further includes balloon 902. Balloon 902 is sealed to the distal part of modified retrieval catheter 13204. In FIGS. 13A and 13C balloon 902 is shown in an inflated form, where it has a shape having a radial symmetry around an x axis and a maximal cross section (along Y-Z plane) of a circle having a diameter of at least 10 mm, or at least 15 mm or at least 20 mm; balloon 902 is dimensioned and configured to occlude a distal point within fallopian tube 154 (e.g., ampulla 158 or infundibulum 157). In the non-inflated form (not shown), balloon 902 may be flat with the dimensions of modified retrieval catheter 13204, such that the cross section around the balloon area may be similar to the cross section along modified retrieval catheter 13204. FIG. 13D shows modified retrieval catheter 13204 in an isometric perspective (balloon 902 is not shown). Modified retrieval catheter 13204 include inflation openings 905. The number of inflation openings 905 is an example, another number of opening may be used. Inflation openings 905 are in fluid connection with a lumen of fluid delivery lumen 904. Balloon 902 may be inflated with liquid via fluid delivery lumen 904 and inflation openings 905. With reference back to FIG. 13A, balloon 902 include exit hole 908; after inflation of balloon 902 with liquid, liquid may be discharged from exit hole 908 into fallopian tube 154 (similar to the example in FIGS. 9A-9B). In an embodiment internal aspiration lumen 208 may have circular cross section (as described above regarding in FIG. 12E).

Device 1300 may be integrated with introductory catheter 202 and be used for oocyte retrieval.

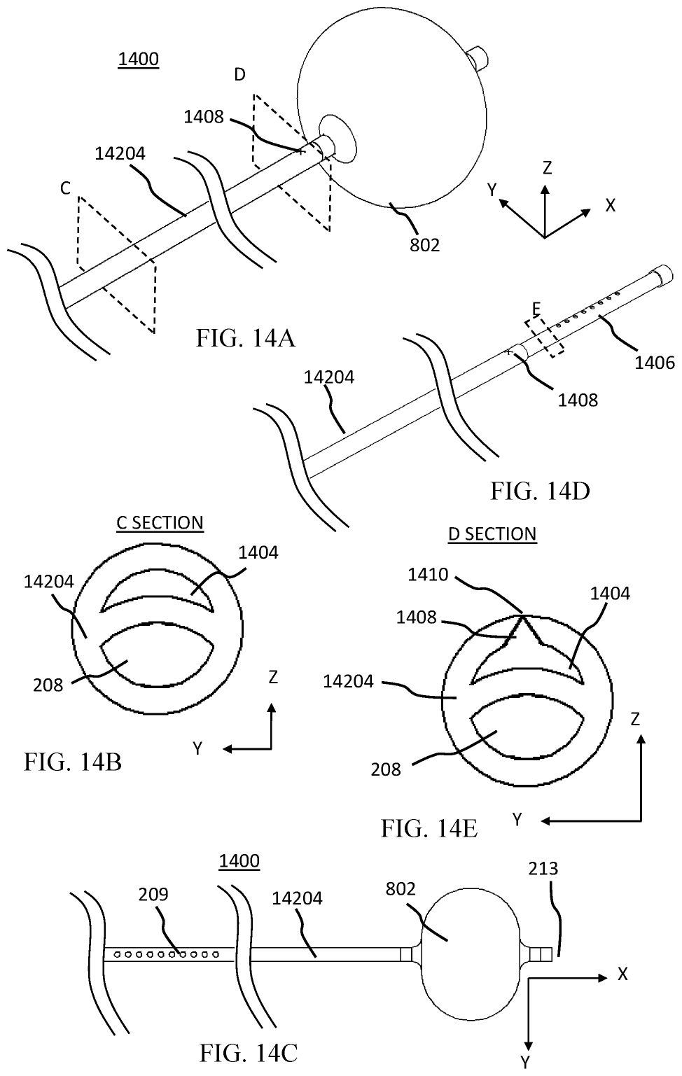

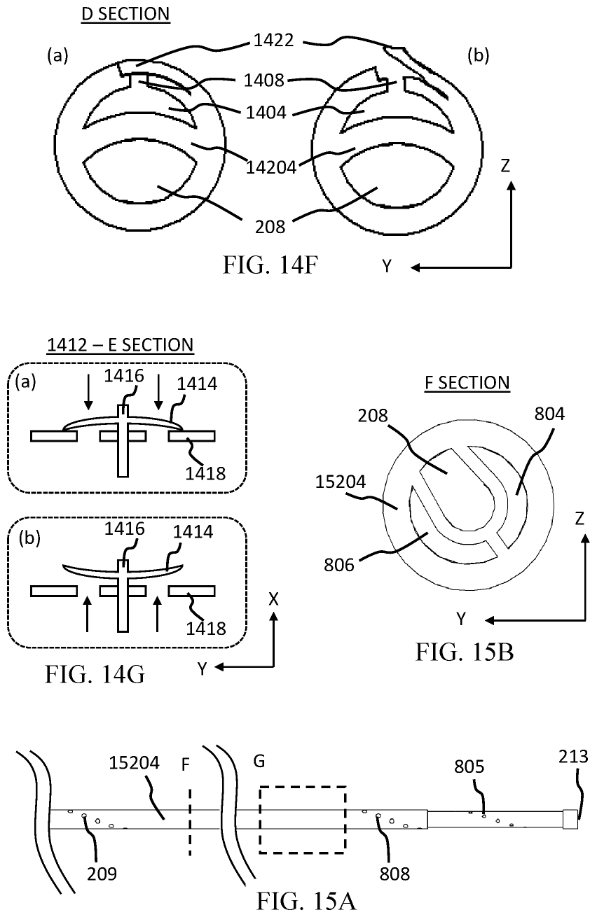

FIGS. 14A-14G show another example of oocyte retrieval device 1400. As in FIGS. 12A-I, FIGS. 14A-14G show coordinate system x-y-z for illustrative purposes only, and does not reflect on the device operation. Device 1400 includes a modified retrieval catheter 14204.

FIG. 14A in an isometric projection. Device 1400 includes modified retrieval catheter 14204, which may be made from a flexible biocompatible material, such as: silicone, polyurethane, Teflon etc. In an example, modified retrieval catheter 14204 may have circular cross section with a diameter of 0.8-1.6 mm, allowing it to pass in the isthmus 159 of female reproductive system. FIG. 14B shows a cross section of modified retrieval catheter 14204 along plane C marked in FIG. 14A. In a nonlimiting example, modified retrieval catheter 14204 may include internal aspiration lumen 208 and/or fluid delivery lumen 1404. In some embodiments, modified retrieval catheter 14204 may include only fluid delivery lumen 1404. In the nonlimiting example, internal aspiration lumen 208 cross section has an incircle with a diameter of at least 0.3 mm or at least 0.4 mm or at least 0.5 mm; in an example, internal aspiration lumen 208 may allow the passage of a guidewire with similar diameter as the incircle of internal aspiration lumen 208 cross section. FIG. 14C shows device 1400 and modified retrieval catheter 14204 from a bottom view. Modified retrieval catheter 14204 further includes Retrieval catheter aspiration openings 209. Retrieval catheter aspiration openings 209 are in fluid communicating with internal aspiration lumen 208; retrieval catheter aspiration openings 209 allow suctioning into internal aspiration lumen 208. In FIG. 14C, eight retrieval catheter aspiration openings 209 are marked as an example, but any number of retrieval catheter aspiration openings 209 may be used (e.g., 1-50 or 1-10). In an example, retrieval catheter aspiration openings 209 are distanced from modified retrieval catheter tip 213 by few centimeters (e.g., 4-8 cm); when positioned inside fallopian tube 154, retrieval catheter aspiration openings 209 are proximal to the ampulla (toward the uterus side) while modified retrieval catheter tip 213 is distal to the ampulla (toward the ovaries). Now in FIGS. 14A and 14C, device 1400 further includes a balloon 802, similar in properties and design to balloon 802 described in above, regarding FIGS. 12A-D and 8A-C. Balloon 802 is sealed to the distal part of modified retrieval catheter 14204. In FIGS. 14A and 14C balloon 802 is shown in an inflated form. In the non-inflated form (not shown), balloon 802 may be flat with the dimensions of modified retrieval catheter 14204, such that the cross section around the balloon area may be similar to the cross section along modified retrieval catheter 14204 (e.g., a circumcircle for the cross section of the deflated balloon may have the same diameter as the cross section of modified retrieval catheter 14204, or a diameter that is larger by no more than 0.2 mm from the diameter of the cross section of modified retrieval catheter 14204). FIG. 14D shows modified retrieval catheter 14204 in an isometric perspective (balloon 802 is not shown). Modified retrieval catheter 14204 include second retrieval catheter openings 1406. The number of fluid delivery openings 1406 is an example, another number of opening may be used (e.g., 1-5 openings, 5-10 openings). Fluid delivery openings 1406 are in fluid connection with fluid delivery lumen 1404. Balloon 802 may be inflated with gas or liquid via fluid delivery lumen 904 and second retrieval catheter openings 1406. Modified retrieval catheter 14204 further includes breach opening 1408, seen in FIGS. 14A and 14D. Breach opening 1408 is located on the surface of modified retrieval catheter 14204, proximal to balloon 802. Breach opening 1408 is in fluid connection with a fluid delivery lumen 1404, as described herein. In use, breach opening 1408 is sealed when modified retrieval catheter 14204 is inserted into the fallopian tube, and before balloon 802 is inflated; once modified retrieval catheter 14204 is in retrieval position, and balloon 802 is fully inflated, breach opening 1408 may be breached (opened, broken), such that liquid can flow from fluid delivery lumen 1404 into fallopian tube 154. Breaching of breach opening 1408 may be permanent (i.e. irreversible for sealing while in use) or reversable.

FIG. 14E shows one example of a cross section of modified retrieval catheter 14204 along plane D, marked in FIG. 14A. According to one example, shown in FIG. 14E, a breaching mechanism may be provided by thinning a thin line 1410 in the external face of modified retrieval catheter 14204. Thus, when supplying a gas or liquid flow to balloon 802, pressure in fluid delivery lumen 1404 increases. When a threshold pressure is reached (e.g., in the region of 1-10 atm or 0.1-1 atm pressure) thin line 1410 may be breached (broken, teared). According to one example balloon 802 may be inflated with liquid at pressure slightly below the threshold pressure. By further pressuring liquid in fluid delivery lumen 1404 above threshold pressure, breach opening 1408 may be breached, thin line 1410 may be broken (not seen); liquid can subsequently exit from breach opening 1408.

According to another example (not shown), a breach mechanism may be provided by applying electrical current in breach opening 1408; electrical current may heat a thin wire threaded along thin line 1410 and break it when heated que to joule heating effect. In an example, thin line may be designed to deform, melt or break at a threshold temperature (for example 50-100 degrees Celsius).

According to another example, a wire (not shown) may be inserter in fluid delivery lumen 1404 (with or without external guidance such as ultrasound imaging) and pierce thin line 1410.

FIG. 14F shows an alternative cross section of modified retrieval catheter 14204 along plane D, marked in FIG. 14A, with yet another example of breach mechanism. According to one example breach mechanism may include a leaf cover 1422 to breach opening 1408. Insert (a) shows leaf cover 1422 closed, while insert (b) shows leaf cover 1422 in the breached (open) position. Leaf cover 1422 may be made from the same material of modified retrieval catheter 14204 or another biocompatible material. In the closed position leaf cover 1422 covers breach opening 1408 from all sides, but it is only connected (attached) to modified retrieval catheter 14204 in part of the circumference of breach opening 1408. Leaf cover 1422 has elasticity which allows it to bend and expose (open) breach opening 1408. In the closed position, leaf cover 1422 keeps pressure breach opening 1408 thus blocking liquid from exiting from fluid delivery lumen 1404 and outside of catheter 14204. Pressure is kept by the spring structure of leaf cover which may be designed to hold pressure up to a threshold pressure (e.g., in the region of 1-10 atm or 0.1-1 atm pressure). According to one example balloon 802 may be inflated with liquid at pressure slightly below the threshold pressure. By further pressuring liquid in fluid delivery lumen 1404 above threshold pressure, leaf cover 1422 may bend to the position shown in FIG. 14F insert (b) and thus breach opening 1408 may be breached.

In some embodiments a plurality of breach openings may exits (e.g. 2-10 breach opening).

FIG. 14G shows an optional cross section in a side view along a cut E marked in FIG. 14D. FIG. 14G shows an unidirectional valve 1412, which may be installed in another fluid delivery lumen 1404 in some examples; the top insert (a) shows unidirectional valve 1412 in closed position, while the bottom insert (b) shows valve 1412 in the open position. In the closed position (a) valve 1412 blocks the flow of liquid in another fluid delivery lumen 1404 and in the open position (b) valve 1412 allows the flow of liquid in another fluid delivery lumen 1404. In one embodiment, Valve 1412 is a one-way valve, which may allow gas or liquid to flow into the distal part of fluid delivery lumen 1404, but not backward (from the distal to the proximal direction). In one example, valve 1412 may be designed with membrane 1414, held in the center by pin 1416; a flow in one direction may bend membrane 1414 to allow entrance of gas or liquid, but a flow in the opposite direction pushes membrane 1414 on wall 1418 and blow gas or liquid passage. Valve 1412 is installed distal to breach opening 1408, but proximal to second retrieval catheter opening 1406. In one example, after inflation of balloon 802 with gas or liquid, and after breach opening is breached, valve 1412 keeps balloon 802 from deflating trough breach opening 1408. When oocyte retrieval procedure is completed, and in order to deflate balloon 802, valve 1412 may be breached by one of several methods. In one example, a wire (not shown) may be inserted in fluid delivery lumen 1404 and break some part of valve 1412, e.g., membrane 1414 or pin 1416 or wall 1418. In one example the wire my simply push membrane 1414 to allow deflation. In another example, electrical current may pass in membrane 1414 and cause it to heat and deform. In one example, suction force may be applied in fluid delivery lumen 1404 causing valve 1412 to bend or break. In one example, a wire (not shown) may be inserted in fluid delivery lumen 1404 and pierce balloon 802 in order to deflate the balloon.

Device 1400 may be integrated with an introductory catheter such as introductory catheter 202 to a device of oocyte retrieval.

According to an example the following method may be used to retrieve oocytes using modified retrieval catheter 14204:

(i) Inserting modified retrieval catheter 14204 into fallopian tube 154 while balloon 802 is not inflated;
(ii) positioning balloon 802 behind ampulla 158 (e.g., in the infundibulum 157);
(iii) inflating balloon 802 to block fallopian tube, e.g., as shown in FIG. 9A, using gas or liquid;
(iv) breaching the breach opening 1408 by any of the method mentioned above (e.g. suppressing liquid pressure in lumen 1404, applying electric current to heat and deform breach opening, piercing by external wire etc.);
(v) flushing liquid into fallopian tube 154 from breach opening 1408, to flush oocytes toward retrieval catheter aspiration openings 209; and
(vi) collecting oocytes into internal aspiration lumen 208 using a suctioning operation.
(vii) Deflating balloon 802.

According to one example, device 1400 allows the inflation of the balloon 802 without spilling of flushing liquid into fallopian tube 154, and device 1400 further allows the spilling of flushing liquid into fallopian tube 154 without deflating balloon 802.

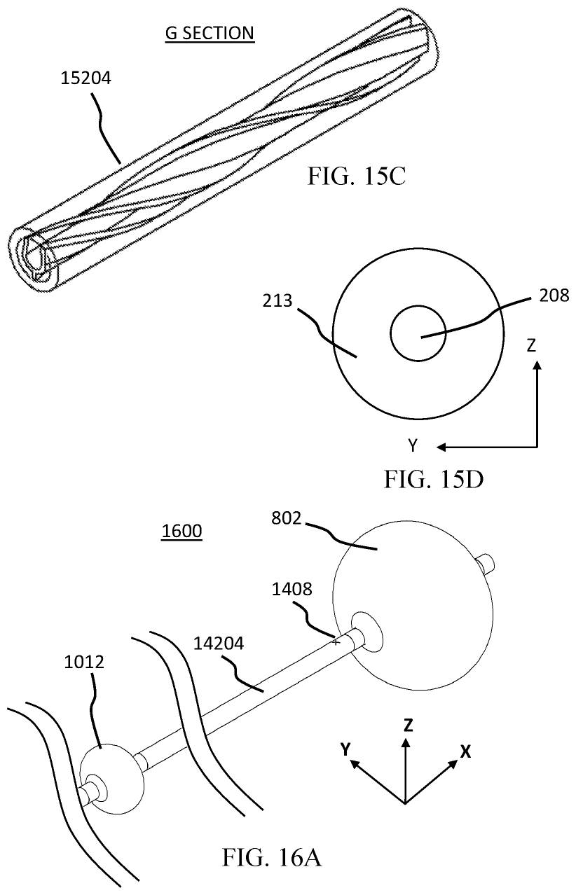

FIGS. 15A-D show another example of modified retrieval catheter 15204. FIG. 15 shows the distal end of modified retrieval catheter 15204 from a side view. FIG. 15B shows a cross section of modified retrieval catheter 15204 along plane F marked in FIG. 15A. FIG. 15C shows a cut along a section G marked in FIG. 15A of modified retrieval catheter 15204 in isometric perspective view; in FIG. 15C the outer layer of modified retrieval catheter 15204 is transparent for readers understanding. FIG. 15D shows retrieval catheter tip 213 of modified retrieval catheter 15204. Modified retrieval catheter 15204 is similar to retrieval catheter 12204 in operation, part numbers, dimensions, etc. Modified retrieval catheter 15204 includes two lumens (e.g., 208 and 904) that are arranged in a helically wound arrangement along a length of the retrieval catheter, to form a spiral and/or helical arrangement internally thereof. In such an arrangement, openings 209, 805, and/or 808 are arranged in a spiral form about an outer sidewall of the retrieval catheter. The arrangement of retrieval catheter aspiration openings 209 from all sides of modified retrieval catheter 15204 may ensure that at least some of retrieval catheter aspiration openings 209 are not in touch with fallopian tube tissue. In some embodiments, the helically wound arrangement defines a hollow passage extending internally along a length of the retrieval catheter, wherein hollow passage is dimensioned to receive a guidewire therethrough. In addition, the design of internal aspiration lumen 208 is such that a guide wire (e.g., a guide wire having a circular cross section) may pass in the center of modified retrieval catheter 15204 and exit from a circular hole in retrieval catheter tip 213 (FIG. 15D). According to one example the following non limiting dimensions may be achieved: external diameter of modified retrieval catheter 15204 of 1.3 mm to 1.5 mm, internal aspiration lumen 208 allowing a guide wire having a cross section with circumcircle of 300-600 um. In some embodiments, any of the previous embodiments of aspiration catheters disclosing two or more internal lumens of the retrieval catheter may be formed such that the internal lumens are wound helically with respect to one another and\or aspiration openings are positioned along different sides of the aspiration catheters.

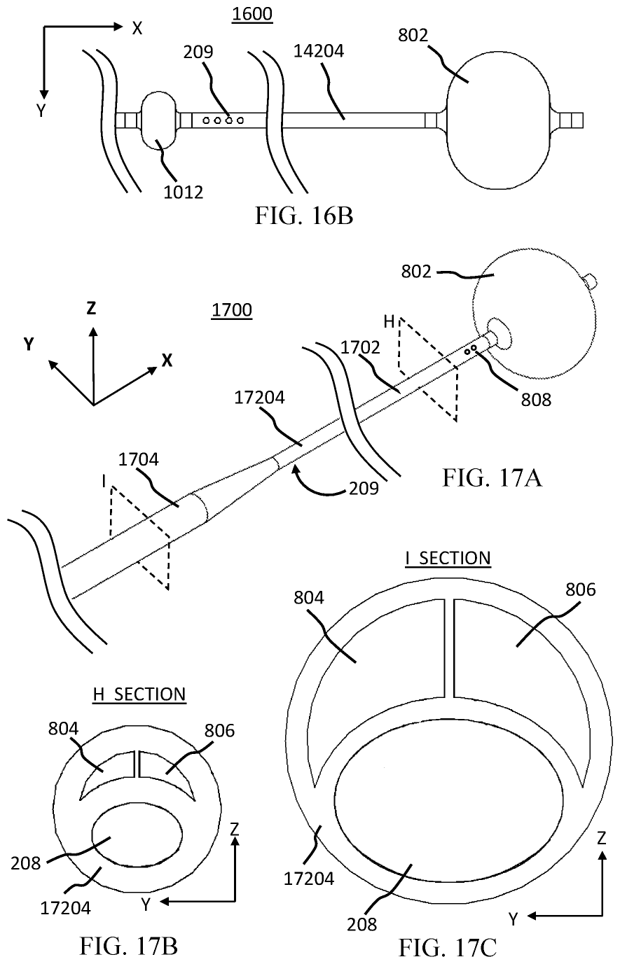

FIGS. 16A-16B show another example of oocyte retrieval device 1600. Device 1600 include any of retrieval catheter previously introduced (FIGS. 16A-16B are demonstrated with modified internal catheter 14204 but generalization to any of retrieval catheters 204, 8204, 9204, etc. is implicit), a first balloon (e.g., 802 or 902) and a second balloon 1012, as exampled in FIGS. 10A-10B. balloon 1012 is connected to inflation lumen 804 by holes (not seen), allowing the inflation of second balloon 1012 at the same time with first balloon 802. FIG. 16A shows device 1600 in isometric perspective, while FIG. 16B shows device 1600 from a bottom view. When inflated, balloon 1012 may occlude fallopian tube 154 such that liquids cannot pass from one side of balloon 1012 to the other side.

FIGS. 17A-C show another example of oocyte retrieval device 1700. Device 1700 includes retrieval catheter 17204 and a first balloon (e.g., 802 or 902). FIG. 17A shows device 1700 in isometric perspective, FIG. 17B shows a cross section of modified retrieval catheter 17204 along plane H marked in FIG. 17A, and FIG. 17C shows a cross section of modified retrieval catheter 17204 along plane I marked in FIG. 17A. Device 1700 is similar to device 800 and modified retrieval catheter 17204 is similar to modified retrieval catheter 8204 (similar numberings on figure indicate similar part numbers and descriptions as to the above), with the following addition: proximal to retrieval catheter aspiration openings 209 (which are not seen but their position is marked by an arrow), retrieval catheter 17204 widen from a distal section having a diameter smaller than 1.4 mm or smaller than 1.6 mm to a proximal section having a diameter larger of 2 mm (e.g. a diameter in the range 2-3 mm or in the range 3-4 mm or in the range 4-5 mm). The section of modified retrieval catheter having lower diameter is referred below as retrieval catheter distal section 1702 and the section of retrieval catheter 17204 having larger diameter is referred below as retrieval catheter proximal section 1704. As seen in FIGS. 17B-17C the number of lumens and positions does not change between the two sections, but the dimensions of each lumen increase. In one example, the design allows for a guide wire to be passed along into internal aspiration lumen 208 without interruption caused by the dimension change. In one example, when modified retrieval catheter is positioned in retrieval position inside female reproductive system, retrieval catheter distal section 1702 is positioned inside fallopian tubes 154 end extending to uterus 153, and retrieval catheter proximal section 1704 is positioned outside of fallopian tubes 154 (e.g., in uterus 153) and extending outside of the patient body. The increase in dimensions reduces the flow resistance of each lumen (the flow resistance is inversely proportional to a cross sectional dimension of the lumen in the power of 4, and linearly proportional to the length of the lumen), and may facilitate or accelerate the flow of gases and liquids in these lumens.

The widening of retrieval catheter presented in FIGS. 17A-C may be implemented implicitly to all the catheters presented above and below, having any number of lumens (e.g. 8204, 9204, 10204, 12204, 13204, etc.)

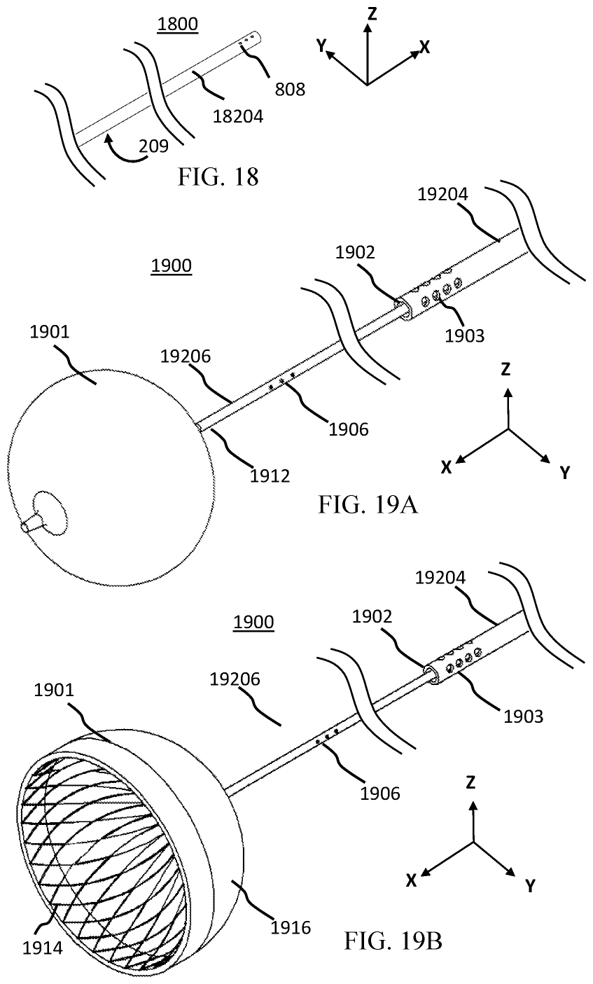

FIG. 18 shows another example of modified internal catheter 18204 in an isometric perspective. Internal catheter 18204 has two lumens, internal aspiration lumen 208 allowing aspiration of oocytes from retrieval catheter aspiration openings 209 and fluid delivery lumen 806 allowing discharge of liquid into the fallopian tubes 154 trough fluid delivery port 808.

In some of the figures above, retrieval catheter aspiration openings 209 is shown proximal to fluid delivery port 808 or breach opening 1408; in such cases flushing direction is from distal to proximal (from ovaries side to uterus side). However, in some examples the position of these opening may be altered (reversed, switched) such that flashing direction may be from the proximal side to the distal side.

FIG. 19A shows another example of oocyte retrieval device 1900 in an isometric perspective. Device 1900 includes retrieval catheter 19204, irrigation catheter 19206, and an occluding element 1901. FIG. 19 shows only the distal end of retrieval catheter 19204 and irrigation catheter 19206. In an example, retrieval catheter 19204 may be made of a flexible biocompatible material such as FEP, PTFE, PEBAX, Teflon, etc. In an example, retrieval catheter 19204 may have a circular external cross section with a diameter of 1 mm-1.6 mm. In an example retrieval catheter 19204 may fit the dimensions of the female reproductive system's isthmus; in use retrieval catheter 19204 may by inserted into the isthmus without creating damage. In one example retrieval catheter 19204 is dimensioned such that it may seal the female reproductive system's isthmus; when inserted into the isthmus fluid from the fallopian tube cannot pass to the uterus. Retrieval catheter 19204 includes a retrieval lumen 1902. In an example retrieval lumen 1902 may have a circular cross section with a diameter smaller from the catheter external diameter by 100-600 um. Optionally, retrieval catheter 19204 may further include one or more aspiration opening(s) 1903 which are in fluid connection with retrieval lumen 1902. In some cases, aspiration openings 1903 may be located on one side of retrieval catheter 19204 circumference. In some cases, aspiration openings 1903 may be located on plurality of sides of retrieval catheter 19204 circumference. In some cases, aspiration openings 1903 may be located on the tip of retrieval catheter 19204.

Irrigation catheter 19206 may be made of a flexible material such as EBAX, Teflon, FEP, PTFE, PEEK, etc. In an example, retrieval catheter 19204 may have a circular external cross section with a diameter of 0.4 mm-0.9 mm. In an example irrigation catheter 19206 may fit inside retrieval lumen 1902. Retrieval catheter 19204 includes irrigation lumen 1904 (not shown). Irrigation catheter further includes one or more irrigation opening(s) 1906 which are in fluid connection with irrigation lumen 1904. In one example irrigation opening 1906 may be open at all times. In one example irrigation openings 1906 may be breach openings, similar to breach opening 1408. In some cases, irrigation openings 1906 may be located on one side of irrigation catheter 19206 circumference. In some cases, irrigation openings 1906 may be located on plurality of sides of irrigation catheter 19206 circumference. In one example irrigation openings 1906 are breach openings, similar in description to breach opening 1408. In an example breach mechanism may be any of the describes above. In some examples, irrigation catheter 19206 may be shifted relative to and along retrieval catheter 19204.

Occluding element 1901 may be fixed (attached, glued) to the distal part of irrigation catheter 19206.

FIG. 19A shows one example in which occluding element 1901 may be a balloon, e.g., a balloon similar in mechanics, dimensions, materials, and\or other considerations to balloon described above (e.g., 802 or 902). In one example, balloon 1901 may be in fluid connection with irrigation lumen 1904. In another example, balloon 1901 may be in fluid connection with another lumen in irrigation catheter 19206 (not seen in the figures). Balloon 1901 may be inflated with gas of liquid from outside.

FIG. 19B shows one example in which occluding element 1901 is a stent 1914 covered with a diaphragm 1916. Stent 1914 may be made for example from an elastic metal such as stainless steel, cobalt chromium alloys, nickel titanium alloy (Nitinol), platinum, tantalum alloys, etc.

When inflated or expended (as demonstrated in FIGS. 19A-19B), Occluding element 1901 may be dimensioned to block fallopian tube 154, such that liquid from one side of balloon 1901 may not pass to the other side of it. In one example Occluding element 1901 may be dimensioned (e.g., inflated, expended) to block a body lumen having a cross section with a dimeter larger than 10 mm or 15 mm or 20 mm. When contracted (e.g. deflated, folded etc., contracted position not seen in the figures) the dimensions of occluding element 1901 may be such that is can fit inside aspiration lumen 1902.

In one example, in which occluding element 1901 is a balloon, device 1900 may include a valve 1912 installed distal to irrigation openings 1906 and proximal to balloon 1901. Valve 1912 may be similar in design and considerations to valve 1412. Valve 1908 is a one-way valve, allowing fluid to go from the proximal to the distal direction, but preventing a flow of fluid from the distal to the proximal direction. In one example, gas or liquid flowing in irrigation lumen 1904 may pass through valve 1912 and inflate balloon 1901. Valve 1912 may then keep balloon 1901 inflated even if pressure in irrigation lumen 1906 drops.

According to an example, the following method may be used to retrieve oocytes using device 1900:

(i) Inserting retrieval catheter 19204 into the fallopian tube isthmus;
(ii) Inserting irrigation catheter 19206 into retrieval catheter 19204 and into fallopian tube distal to the ampulla;
(iii) Inflation of balloon 1901 to occlude fallopian tube;
(iv) Irrigation of fallopian tube 154 from irrigation opening(s) 1906 to flush oocytes toward aspiration opening(s) 1903;
(v) Collecting oocytes into retrieval lumen 1902 using suction operation;
(vi) Deflating balloon 1901;
(vii) retracting all elements from the fallopian tube.

According to another example, the following method may be used to retrieve oocytes using device 1900:

(i) Inserting retrieval catheter 19204 into the fallopian tube, advancing retrieval catheter 19204 distal to the ampulla;
(ii) Inserting irrigation catheter 19206 into retrieval catheter 19204, advancing irrigation catheter 19206 until retrieval catheter 19204 tip;
(iii) Pulling retrieval catheter 19204 proximal to the ampulla, while leaving irrigation catheter 19206 distal to the ampulla.
(iv) Expanding occlusion element 1901 to occlude fallopian tube;
(v) Irrigation of fallopian tube 154 from irrigation opening(s) 1906 to flush oocytes toward aspiration opening(s) 1903;
(vi) Collecting oocytes into retrieval lumen 1902 using suction operation; (vii) Contracting occlusion element 1901;
(viii) Retracting all elements from the fallopian tube.

In some examples, both methods [00161] and [00162] presented may be assisted by internal cameras such as 402 or 502 described above or by external imaging system (e.g. US, X-ray, etc.).

In some examples, insertion of retrieval catheter 19204 into fallopian tube may be assisted by a guide wire. The guide wire may be removed prior to the insertion of irrigation catheter 19206 into retrieval catheter 19204.

In some examples the assignment of aspiration and irrigation catheters and lumen may exchange between them, namely, irrigation of fallopian tube may be done by the exterior catheter, flushing liquid may flow from proximal to distal direction in the fallopian tube, and aspiration may be done using the internal catheter.

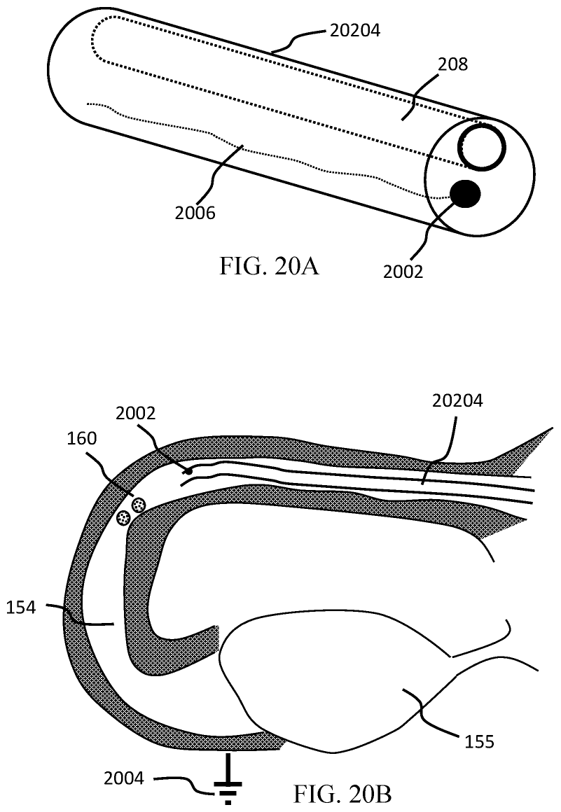

FIGS. 20A-B show another example of retrieval device 2000. FIG. 20A shows a modified retrieval catheter 20204 distal end. Modified retrieval catheter 20204 is similar to retrieval catheter 204 with the following additions: electrode 2002 is positioned on the tip of modified retrieval catheter 20204. Electrode 2002 may be made of metallic biocompatible material (e.g., gold, platinum, etc.). An electrical wire 2006 is connected to electrode 2002 and pass along modified retrieval catheter 20204 and outside of female reproductive system (not shown), where it is connected to electric potential (not shown), relative to female reproductive system. FIG. 20B shows part of female reproductive system and modified retrieval catheter 20204 in retrieval position 252. Female reproductive system is grounded (mark 2004 in FIG. 20B). Grounding of female reproductive system may be done by grounding patient body which, in turn, may be done by wearing electric conductive wrist band on patient hand or leg, or by any similar method. Upon activation of electric potential V on electrode 2002, an electric field is formed in the fallopian tube in conjunction with near electrode 2002. Oocytes 160 which have an electric dipole will be pulled toward the electrode 2002 due to dielectrophoretic force and sequentially collected using suction from retrieval catheter aspiration openings 209, located close to electrode 2002. Application of electric potential on electrode 2002 may be constant (DC current), pulsed, or alternate current (AC). According to an example, the following method may be used to retrieve oocytes:

(i) Grounding patient's body;
(ii) inserting modified retrieval catheter 20204 into fallopian tube 154;
(iii) applying an electric potential to electrode 2002; and
(iv) collecting oocytes into internal aspiration lumen 208 using suction operation.

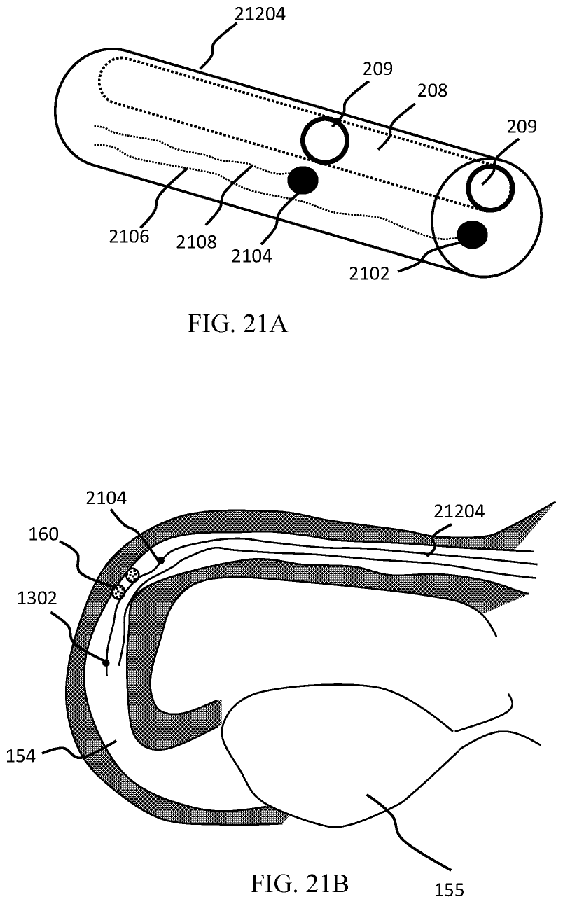

FIGS. 21A-B show another example of retrieval device 2100. FIG. 21A shows a modified retrieval catheter 21204 distal end. Modified retrieval catheter 21204 is similar to retrieval catheter 204 with the following additions: modified retrieval catheter 21204 includes two electrodes 2102 and 2104. Electrode 2102 is positioned at a distal tip of modified retrieval catheter 204, whereas electrode 2104 is positioned few mm (0.5-3) proximally of the tip of modified catheter 21204. Electrodes 2102-2104 may be made of any metallic biocompatible material (e.g., gold, platinum, etc.). Two electrical wires 2106 and 2108 are connected to electrodes 2102 and 2104, respectively. Electrical wires 2106 and 2108 pass along modified retrieval catheter 21204 and outside of female reproductive system (not shown), where they are connected to an electric power source (not shown), an electric field is formed in the fallopian tube by electrodes 2102 and 2104.

FIG. 21B shows part of female reproductive system and modified retrieval catheter 21204 in retrieval position 252. In FIG. 21B, oocytes 160, which have an electric dipole, will be pulled toward the electrodes 2102 and\or 2104 due to dielectrophoretic force and sequentially collected using suction from two or more retrieval catheter aspiration openings 209 located near both electrodes 2102 and 2104. Application of electric potential on electrodes 2102 and 2104 may be constant (DC current), pulsed, or alternate current (AC). According to an example, the following method may be used to retrieve oocytes:

(i) Inserting modified retrieval catheter 21204 into fallopian tube 154;
(ii) applying electric voltage drop between electrodes 2102 and 2104; and
(iii) collecting oocytes into internal aspiration lumen 208 using suction operation.

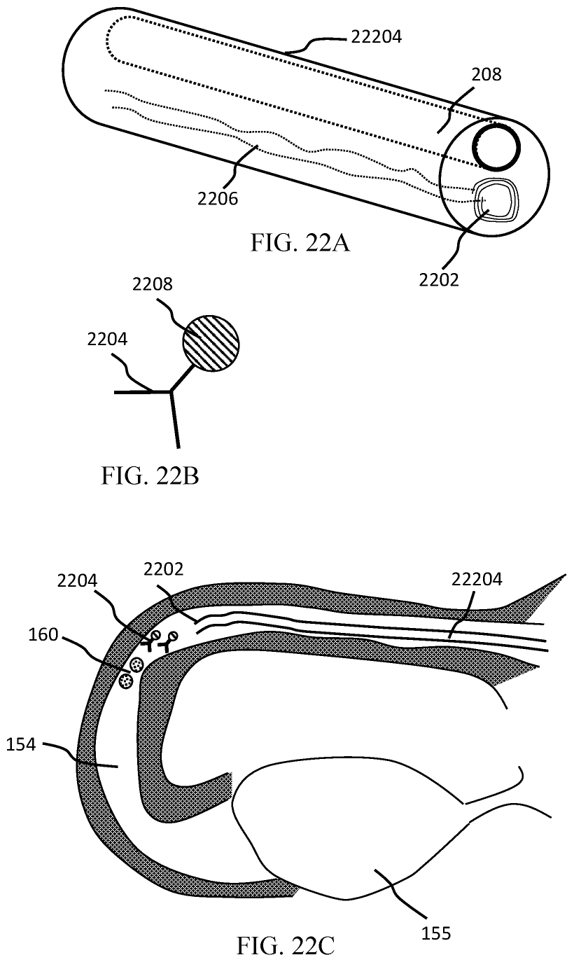

FIGS. 22A-C show another example of retrieval device 2200. FIG. 22A shows a modified retrieval catheter 22204 distal end. Modified retrieval catheter 22204 is similar to retrieval catheter 204 with the following additions: Electromagnet 2202 is positioned at a tip of Modified retrieval catheter 22204. Electromagnet 2202 may be made, for example, from coil windings of metallic (e.g. copper, gold etc.) wire with a possibility of some ferromagnetic material (e.g., ferrite, iron, cobalt etc.) at the core of the coil. Two electrical wires 2206 pass along modified retrieval catheter 22204 and outside of female reproductive system (not shown), where they are connected to a current source. Electromagnet 2202 may receive current from two electrical wires 2206; driving current in coil 2202 creates a magnetic field. FIG. 22B show engineered antibody 2204. Engineered antibodies 2204 are designed to attach to oocytes 160 external membrane. Engineered antibodies 2204 may be attached to ferromagnetic bids 2208 in the lab. Ferromagnetic bids 2208 may be made from several ferromagnetic materials (e.g., ferrite, iron, cobalt, etc.), and may be covered with biocompatible material such as silicone, gold, platinum, etc. FIG. 22C shows part of female reproductive system, modified retrieval catheter 22204 in retrieval position 252, and engineered antibodies 2204 near oocytes 160. According to an example the following method may be used to retrieve oocytes:

(i) Injecting engineered antibodies 2204 into the fallopian tube 154 from modified retrieval catheter 22204. In some embodiments, engineered antibodies 2204 may be injected into uterus 153, wherein some of which will travel into the fallopian tube 154;
(ii) waiting for engineered antibodies 2204 to attach to oocytes 160;
(iii) inserting modified retrieval catheter 22204 into fallopian tube 154;
(iv) applying electrical current to electromagnet 2202; and
(v) waiting for oocytes to be pulled to electromagnet 2202, for collecting.

The devices presented above may be used for purposes other than oocytes retrieval. According to one example, device 200 (with all the variations presented) may be used for cell collections from within the fallopian tube. Cells collected from the fallopian tube may be used for ovarian cancer detection.

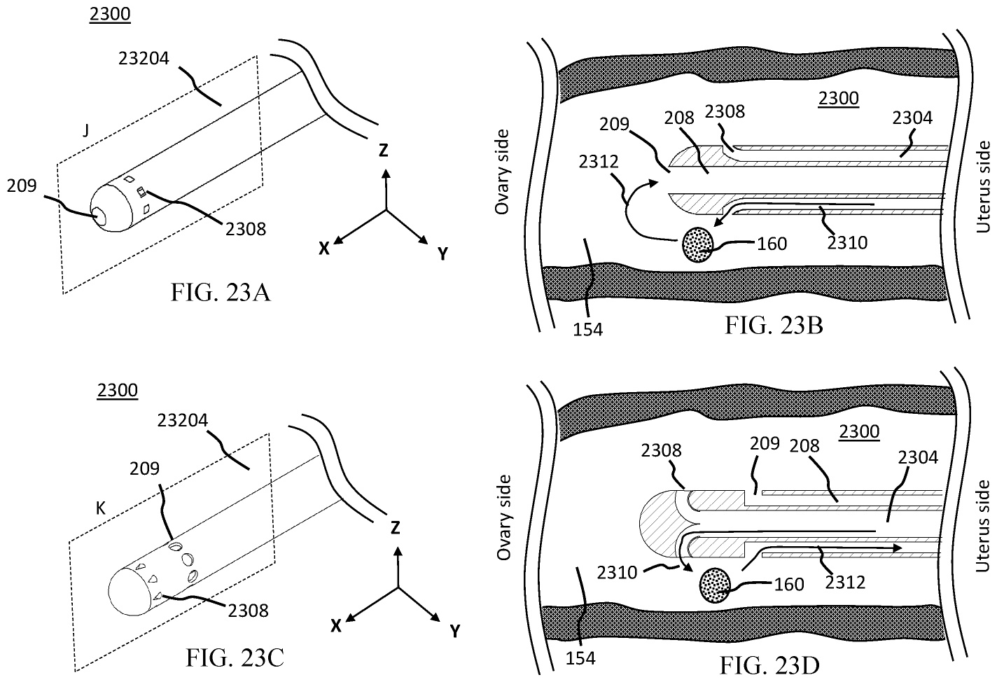

FIGS. 23A-23D show another example of retrieval device 2300. FIG. 23A shows a modified retrieval catheter 23204 distal end. FIG. 23B shows modified retrieval catheter 23204 distal end cut along section J marked in FIG. 23A, positioned inside a section of fallopian tube 154, side by side with oocyte 160. FIG. 23C shows an alternative option for modified retrieval catheter 23204 distal end. FIG. 23D shows alternative option for modified retrieval catheter 23204 distal end cut along section K marked in FIG. 23C, positioned inside a section of fallopian tube 154, side by side with oocyte 160.

Modified retrieval catheter 23204 is designed to fit inside the fallopian tube 154. In an example modified retrieval catheter 23204 has a circular cross section with a diameter in the range of 1.1-1.6 mm. Modified retrieval catheter 23204 includes an internal aspiration lumen 208. In one example (FIGS. 23A-B) retrieval catheter lumen 208 has a circular cross section with a diameter of at least 100 um (in an example 400-900 um). Internal aspiration lumen 208 is in fluid connection with retrieval catheter aspiration opening(s) 209. Retrieval catheter aspiration opening(s) 209 may be located on the tip (most distal end) of modified retrieval catheter 23204 (FIGS. 23A-B) or on the circumference of modified retrieval catheter 23204 (FIGS. 23C-D). Modified retrieval catheter 23204 further includes a fluid delivery lumen 2304. In one example (FIG. 23A-B) fluid delivery lumen 2304 having a ring cross section. In one example Fluid delivery lumen 2304 surrounds internal aspiration lumen 208 from all sides. In another example (FIG. 23C-D) fluid delivery lumen 2304 having a circular cross section. Fluid delivery lumen 2304 is in fluid connection with one or more irrigation openings 2308. In one example, irrigation openings 2308 are located on the circumference of modified retrieval catheter 23204, few millimeters proximal or distal to retrieval catheter aspiration opening 209 (e.g., 5-25 mm). In an example, 4-12 irrigation openings 2308 are equally spaced in a circular pattern from all sides of modified retrieval catheter 23204, According to one example, liquid may be irrigated from fluid delivery lumen 2304 to irrigation openings 2308 and outside of retrieval catheter 23204, as demonstrated by arrow 2310 in FIGS. 23B and 23D. Irrigation may be sourced by a liquid source connected to modified retrieval catheter 23204 back (proximal end), which is not seen in FIGS. 23A-D, but can be learned from description above of FIGS. 8-11, for example. According to one example, liquid may be aspired from outside of retrieval catheter 23204 trough retrieval catheter aspiration opening 209 and into internal aspiration lumen 208, as demonstrated by arrow 2312 in FIGS. 23B and 23D. Aspiration can be operated using a pump connected to modified retrieval catheter 23204 back (proximal end), which is not seen in FIGS. 23A-D, but can be learned from description above of FIGS. 7-11, for example.

According to one example the insertion of modified retrieval catheter 23204 into fallopian tube 154 is accompanied by continuous irrigation from irrigation openings 2308 and continuous aspiration from retrieval catheter aspiration opening 209.

According to one example, the following method may be used to retrieve oocytes from fallopian tube 154 in a transcervical procedure:

Irrigate flushing liquid and aspirate same flushing liquid in parallel to the insertion of modified retrieval catheter 23204 into fallopian tube 154, from the proximal end to the distal end.

This method may be advantageous in few aspects: (1) it may facilitate the insertion and navigation of the catheter into the lumen. (2) it may help washing oocytes 160 from fallopian tube 154 to aspiration opening 209. According to another example, the following method may be used to retrieve oocytes from fallopian tube 154 in a transcervical procedure:

a) Insert modified retrieval catheter 23204 to the fallopian tube (e.g., distal to the ampulla 158)
b) Irrigate flushing liquid and aspirate same flushing liquid in parallel to the pullback of modified retrieval catheter 23204 from fallopian tube 154, from the distal end to the proximal end.

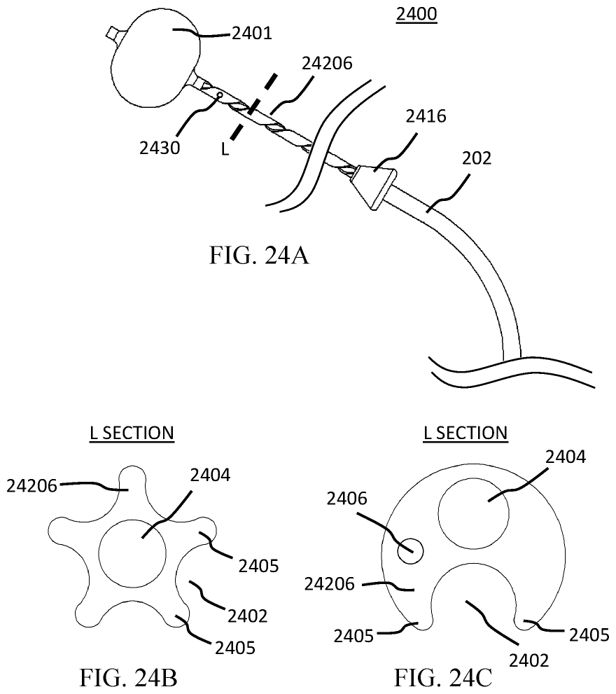

FIGS. 24A-24C show another example of retrieval device 2400. FIG. 24A shows in a side view the distal end of device 2400. device 2400 includes introduction catheter 24202. Introduction catheter 24202 may be similar to any of introduction catheters presented in this disclosure (e.g. 202, 4202a, 4202b, 11202, etc.). Introduction catheter 24202 may be designed to enter the uterus of a female patient in a transcervical procedure. Introduction catheter 24202 may be made from a biocompatible material. Introduction catheter 24202 has dimensions allowing it to enter the cervix of a female patient, e.g., a woman. In one example introduction catheter 24202 has a cross section with a circumcircle diameter smaller than 5 mm or 3 mm. Introduction catheter 24202 includes introductory catheter lumen 206, similar to introduction catheter lumen 206 described in FIG. 2A, which is not seen in FIG. 23A. Introductory catheter lumen 206 may be designed to host retrieval catheter 24204 described below or irrigation catheter 19206, or any of retrieval catheters described in this disclosure (204, 8204, etc.)

Device 2400 may include funneling element 2416, installed on the tip of introduction catheter 24202. In one example funneling element 2416 may be designed for funneling liquids coming out of fallopian tube 154 toward the uterus side into introductory catheter lumen 206. In one example funneling element 2416 may be designed for occluding liquids coming out of fallopian tube 154 from entering uterus 153. In one example funneling element 2416 may be similar to funneling element 1116 described above. In one example funneling element 2416 may be made from a soft material (e.g., silicone, rubber, Teflon) and may be conically shaped. In one example funneling element 2416 may be an inflatable balloon. In one example funneling element 2416 may designed to be wedged in the uterotubal junction or in the isthmus.

Device 2400 may further include retrieval catheter 24204. Retrieval catheter 24204 is designed to enter inside fallopian tube 154. In one example Retrieval catheter 24204 has a cross section with a circumcircle diameter smaller than 1.7 mm or 1.5 mm or 1.3 mm. FIGS. 24B and 24C show two non-limiting examples of optional cross sections of retrieval catheter 24204 along cut L seen in FIG. 24A. Retrieval catheter 24204 includes fluid delivery lumen 2404 for providing the flushing fluid. In one example fluid delivery lumen 2404 may have a cross section having an incircle diameter of at least 300 um or at least 500 um. Fluid delivery lumen is in fluid connection with irrigation port 2430 (or a plurality of irrigation ports). Retrieval catheter 24204 further includes conduit 2402 or plurality of conduits 2402 for allowing the flushed fluid delivered from fluid delivery lumen 2404 to return towards the uterus or introduction catheter 24202. In the example in FIG. 23B there are 5 conduits 2402 and in the example in FIG. 23C there is one conduit 2402. Any number of conduits (e.g. 1-10) is possible. The discussed herein with respect to a single conduit is applicable for any number of conduits. Conduit 2402 may have a cross section having an incircle with diameter of at least 100 um or at least 300 um or at least 600 um. In an example, when inserted into fallopian tube 154 conduit 2402 when engaging with the inner layer of fallopian tube 154 may serve as a full lumen for the flow of liquid from the fallopian tube toward the uterus. In an example, support arms 2405 in the two sides of conduit 2402 may form support structure to support fallopian tube 154 from collapsing into open lumen 2402. In one example, fluid delivery lumen 2404 and\or conduit 2402 and\or support arm(s) 2405 may be arranged in a helically wound arrangement along a length of the retrieval catheter, to form a spiral and/or helical arrangement internally thereof. The helical arrangement may help to support tissue of fallopian tube 154 from collapsing into open lumen 2402. In some example fluid delivery lumen 2404 and\or conduit 2402 and\or support arm(s) 2405 may not be arranged in a helically wound arrangement along a length of the retrieval catheter According to an example, retrieval catheter 24204 comprise support structure allowing flushing liquid to flow in the fallopian tube toward the uterus.

Device 2400 may further include occluding element 2401, designed to occlude fallopian tube distal to the ampulla from the flow of liquid. Occluding element 2401 may be similar to other occluding elements described herein (e.g., 802, 1901, etc.). In one example occluding element 2401 may be a balloon. In an example, where occluding element 2401 is a balloon, it may be inflated from an optional inflation lumen 2406 seen in FIG. 24C. In another example, where occluding element 2401 is a balloon, it may be inflated from fluid delivery lumen 2404. According to one example, device 2400 allows the inflation of the balloon 2401 without spilling of flushing liquid into fallopian tube 154, and device 2400 further allows the spilling of flushing liquid into fallopian tube 154 without deflating balloon 2401. In FIG. 24A occluding element is seen open (inflated), but it may be closed (deflated). In the closed position occluding element is designed to fit in introduction catheter lumen 206 and in the proximal part of fallopian tube 154. In some examples device 2400 may further include other components described in this disclosure such as: breach opening(s) 1408, valve 1412, etc.

In one example the following method may be used to retrieve oocytes using device 2400:

a) Inserting introduction catheter 24202 into the uterus from the cervix.
b) Inserting retrieval catheter 24204 into the fallopian tube through introduction catheter lumen 206.
c) Occluding fallopian tube distal to the ampulla using occluding element 2401.
d) Irrigating flushing liquid from liquid source (not seen) trough fluid delivery lumen 2404 and irrigation port 2430 into fallopian tube 154 (not seen).
e) Flushing oocytes in fallopian tube toward the uterus with flushing liquid.
f) Funneling flushing liquid by funneling element 2416 into introduction catheter lumen 206.
g) Operating suction in introduction catheter lumen 206 to collect flushing liquid and\or oocytes entrained within (e.g., using a pump, not seen in FIGS. 23A-23C).

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

In the description and claims of the application, each of the words “comprise” “include” and “have”, and forms thereof, are not necessarily limited to members in a list with which the words may be associated. In addition, where there are inconsistencies between this application and any document incorporated by reference, it is hereby intended that the present application controls.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. An oocytes or ovum retrieval device comprising:
a retrieval catheter dimensioned for transvaginal insertion into a cervical canal and for advancing distally into a fallopian tube and the ampulla of a female mammal,
wherein said retrieval catheter comprises:
(i) one or more fluid delivery lumens having a fluid delivery port for delivering a flushing fluid into said fallopian tube, and
(ii) at least one aspiration opening in fluid connection with an internal aspiration lumen of said retrieval catheter, wherein the size of the at least one aspiration opening is determined based on a size of oocytes or ovum.

2. The oocytes or ovum retrieval device of claim 1, further comprising an introductory catheter dimensioned for transvaginal insertion into a cervical canal of a female mammal, wherein said retrieval catheter is dimensioned for slidably advancing within a lumen of said introductory catheter along a longitudinal axis thereof, such that a distal end of said retrieval catheter extends distally of a distal tip of said introductory catheter and into said fallopian tube.

3. (canceled)

4. The oocytes or ovum retrieval device of claim 1, further comprising a first occluding element for deployment at an occlusion point, wherein said first occlusion element is configured to prevent passage of said fluid distally of said occlusion point in the fallopian tube.

5. The oocytes or ovum retrieval device of claim 4, further comprising a second occluding element for deployment at a second occlusion point proximally of said at least one fluid delivery port, wherein said second occlusion element is configured to prevent passage of said fluid proximally of said second occlusion point in the fallopian tube.

6. (canceled)

7. (canceled)

8. The oocytes or ovum retrieval device of claim 1, further comprising one or more fluid reservoirs in fluid connection with said one or more fluid delivery lumens.

9. The oocytes or ovum retrieval device of claim 1, further comprising a vacuum source in fluid connection with said internal aspiration lumen.

10. The oocytes or ovum retrieval device of claim 1, wherein an outer diameter of at least a distal portion of a sidewall of said retrieval catheter is between 1 and 1.4 mm

11.-13. (canceled)

14. The oocytes or ovum retrieval device of claim 1, wherein a ratio between a cross sectional dimension of said internal aspiration lumen and a cross sectional dimension of said fluid delivery lumen is at least 2 to 1.

15. The oocytes or ovum retrieval device of claim 1, wherein said retrieval catheter comprises a plurality of said aspiration openings arranged about an external sidewall of said retrieval catheter.

16. (canceled)

17. (canceled)

18. The oocytes or ovum retrieval device of claim 4, wherein said retrieval catheter further comprises an inflation lumen, and wherein said inflation lumen is in fluid connection with at least one of said first occluding element and said second occluding element.

19. The oocytes or ovum retrieval device of claim 4, wherein said first occluding element comprises at least one fluid delivery port for delivering a flushing fluid into said fallopian tube.

20. The oocytes or ovum retrieval device of claim 1, wherein said one or more retrieval catheters comprises a pressure beaching internal lumen, and wherein said pressure beaching internal lumen is configured to breach an opening is said sidewall of said retrieval catheter when a threshold pressure is reached within said second internal lumen.

21. The oocytes or ovum retrieval device of claim 6, wherein said fluid delivery lumen comprises an internal unidirectional valve to prevent passage of fluid in one direction of said fluid delivery lumen.

22. A method for retrieval of oocytes or ovum from a subject comprising:
providing a device comprising:
a retrieval catheter dimensioned for transvaginal insertion into a cervical canal and for advancing distally into a fallopian tube and the ampulla of a female mammal,
wherein said retrieval catheter comprises:
(i) one or more fluid delivery lumens, each having a fluid delivery port for delivering a flushing fluid into said fallopian tube, and
(ii) at least one aspiration opening in fluid connection with an internal aspiration lumen of said retrieval catheter, wherein the size of the at least one aspiration opening is determined based on a size of oocytes or ovum;
advancing said retrieval catheter distally into said fallopian tube and said ampulla;
delivering a flushing fluid through said distal fluid delivery port into said fallopian tube; and
aspirating said flushing fluid and entrained oocytes or ovum therein through said at least one aspiration opening.

23. The method of claim 22, further comprising:
occluding a passage of said fluid, distally from said fluid delivery port, by inserting a first occluding element, included in the device, for deployment of a first occlusion point in the fallopian tube.

24. (canceled)

25. An oocytes or ovum retrieval device comprising:
a retrieval catheter dimensioned for transvaginal insertion into a cervical canal and for advancing distally into a fallopian tube and the ampulla of a female mammal, wherein said retrieval catheter comprises one or more fluid delivery lumens having a fluid delivery port for delivering a flushing fluid into said fallopian tube, and
an introductory catheter dimensioned for entering a uterus of a woman or a female mammal transcervicaly, wherein the introductory catheter comprises an introductory catheter lumen and at least one aspiration opening in fluid connection with said introductory catheter lumen, wherein the retrieval catheter may be inserted in the introductory catheter lumen, and wherein the introductory catheter lumen allows the aspiration of said flushing fluid.

26. The oocytes or ovum retrieval device of claim 25, wherein the retrieval catheter is inserted in the aspiration catheter lumen.

27. (canceled)

28. The oocytes or ovum retrieval device according to claim 25, further comprising a first occluding element for deployment at an occlusion point, wherein said first occlusion element is configured to prevent passage of said fluid distally of said occlusion point in the fallopian tube.

29. The oocytes or ovum retrieval device of claim 28, wherein at said first occluding element is an inflatable balloon.

30.-34. (canceled)

35. The oocytes or ovum retrieval device of claim 25, wherein the retrieval catheter further comprising a conduit allowing flushing liquid to flow in the fallopian tube toward the uterus.

36. (canceled)
